(19) United States
(12) Patent Application Publication
(10) Pub. No.: US 2008/0221303 A1
(43) Pub. Date: Sep. 11, 2008

(54) Method for the Preparation of Peptide-Oligonucleotide Conjugates

(76) Inventors: Jehoshua Katzhendler, Jerusalem (IL); Yakir Klauzner, Jerusalem (IL); Irena Beylis, Tel Aviv (IL); Michael Mizhiritskii, Rehovot (IL); Yaacov Shpernat, Kiriat-Ono (IL); Boris Ashkenazi, Rehovot (IL); Dmitri Fridland, Ashdod (IL)

Correspondence Address: WINSTON & STRAWN LLP;PATENT DEPARTMENT, 1700 K STREET, N.W., WASHINGTON, DC, 20006, US

(21) Appl. No.: 10/590,000

(22) PCT Filed: Feb. 17, 2005
(86) PCT No.: PCT/IL05/00204

§ 371 Date: Aug. 30, 2007

(30) Foreign Application Priority Data
Feb. 18, 2004 (US) 60/545173

(51) Int. Cl. C07K 19/00

(52) U.S. Cl. 530/323

(57) ABSTRACT

The present invention relates to the synthesis of peptide-oligonucleotide conjugates (POC). More specifically, the invention relates to a novel method for the preparation of peptide-oligonucleotide conjugates, which can be conducted under mild conditions on solid support, can be performed manually or by a synthesizer, can be used to synthesize alternating sequences of peptides and oligonucleotides, and is applicable to the synthesis of a wide variety of peptide-oligonucleotide conjugates constructed from alternate peptide and oligonucleotide blocks.

### FIELD OF THE INVENTION

The present invention relates to the synthesis of peptide-oligonucleotide conjugates (POC). More specifically, the invention relates to a novel method for the preparation of peptide-oligonucleotide conjugates, which can be conducted under mild conditions on solid support, can be performed manually or by a synthesizer, can be used to synthesize alternating sequences of peptides and oligonucleotides, and is applicable to the synthesis of a wide variety of peptide-oligonucleotide conjugates constructed from alternate peptide and oligonucleotide blocks.

### BACKGROUND OF THE INVENTION

Oligomeric bioconjugates, i.e. oligonucleotides, peptides or oligosaccharides bearing unnatural organic structures of constituents of other biopolymers, have during the past two decades found an increasing number of applications in therapeutics and as research tools for molecular and cell biology. Conjugate groups are aimed at providing the oligomeric biomolecules with novel properties, such as altered hydrophobicity or bioaffinity, improved cellular permeation and intracellular delivery, fluorescence, emission, catalytic activity, resistance towards biodegradation or ability to carry metal ions.

For example, peptides can be used to improve the cellular permeability of oligodeoxynucleotides (ODN) used in antisense therapeutic applications. The selective inhibition and expression of specific genes by ODN via antisense technology is an attractive approach to therapeutic drug design.^{1,2} Antisense ODN should have at least two characteristic features: a) rapid cell permeation; and b) stability against nuclease degradation. One strategy to improve intracellular delivery of ODN (DNA) is by using several types of short peptides such as fusogenic, hydrophobic and amphiphilic peptides,³⁻¹⁶ antennapedia third helix homeodomain peptides,^{17,18} NLS type (cationic) peptides,^{19,20} signal peptides,^{16,21} receptor mediated peptides such as RGD,²²⁻²⁴ and pH-dependent endocytosis-mediated peptides.²⁵ In this latter category are included histidine rich peptides²⁶⁻²⁹ and peptides containing the KDEL⁵ or GALA³⁰ motifs. In addition, a new motif of small peptide (SPRK)₄ or SPRR was found to bind to A/T rich sites. Some examples of intracellular translocation of small peptides are the basic residues (47-57) of Tat protein,³¹ residues (267-300) of VP22,³² residues of antennapedia homeodomain, transportan-27 aminoacid long,³³ Penetrain-16 aminoacid long,³⁴ and SV40-7 residues. In addition, MTS has been shown to act as delivery vehicles for drugs as doxorubicin,^{35,36} cyclosporin A,³⁷ metalloporphyrin,¹⁵ imaging agents,³⁸ and ODN.³⁹⁻⁴¹ There are various other examples of cell permeating peptides in the art.⁴²⁻⁶⁷

Synthetic methodologies for the preparation of peptides are well established. There are two major methods of solid phase peptide synthesis that are routinely implemented: the t-Boc approach and Fmoc approach. In the t-Boc approach, the α-amine is protected by t-Boc that is cleaved by treatment with trifluoroacetic acid (TFA). Under these conditions, the side chain protecting groups are stable. Strong acids such as HF or TMSA implement cleavage from the resin (together with side chain protecting groups). In the N^{α}-9-fluorenylmethoxycarbonyl (Fmoc) approach, the α-amino group of the amino acids (AA) is protected by Fmoc that can be cleaved by treatment with piperidine via a β-elimination route. The cleavage of the side chain protecting groups and cleavage from the resin take place by treatment with TFA.

Synthetic methodologies for the preparation of oligonucleotides are also well established. There are three methods of solid-phase oligonucleotide synthesis: (a) the phosphate approach, (b) the phosphite approach, and (c) the H-phosphonate approach. Whereas in the phosphate approach one is required to use coupling reagents in order to form an active phosphate, in the phosphite approach the phosphite is already activated. In the H-phosphonate method, a bond formation between two nucleosides is implemented via an oxidative addition reaction.

Although the synthetic methodologies for the preparation of peptides and oligonucleotides are well known and are currently successfully implemented, they are not fully compatible with the peptide-oligonucleotide hybrid synthesis, since the chemistries used for peptide and DNA synthesis are not fully compatible. The major obstacle of synthesis of peptide-ODN conjugates emanate from the inadequacy of peptide deprotection methods with ODN stability.

While the early syntheses of POCs have mainly been carried out in solution, an increasing number of such conjugates are currently prepared either entirely on a solid support or the conjugate group is introduced upon cleavage of the oligomer from the support. Solid support synthesis is preferred since it is less laborious, most of the side products may be removed by simple washing when the conjugate is still anchored to the support and, after release into solution, only one chromatographic purification is usually needed. The advantages of solid support are especially noticed when a conjugate of two different biomolecules is synthesized, as no purification of the presynthesized oligonucleotide or peptide is necessary. Another attractive feature is the exploitation of a fully automatic machine-assisted synthesis, which allows the convenient preparation of conjugate libraries.

There are two different approaches that have been studied extensively for preparing POCs. The first is the sequential (or stepwise) synthesis and the second is the fragmental conjugation.

In the sequential synthesis, the peptide and oligonucleotide are synthesized sequentially on automatic synthesizers. For peptide synthesis, Fmoc chemistry has been used most frequently, as its reaction conditions are milder than for Boc chemistry. In various studies, the peptide was usually assembled first on the solid support, followed by oligonucleotide synthesis. Various Peptide—oligonucleotide syntheses by stepwise methods are described in the literature.^{43, 47, 68-79}

Sequential synthesis of POCs according to current methods has several limitations. Specifically, known methods are restricted to pairs of peptide-ODN: one starts from the oligonucleotide and adds the peptide or vice versa. However, no one has developed a general method that allows several alternating sequences. In addition, synthetic methods that employ Boc protecting groups require that the synthesis is started from the peptide site, since cleavage from the resin by this method involves the use of a strong acid. In the case of synthetic methods which employ Fmoc protecting groups, there is the possibility to start the synthesis either from the peptide side or from the oligonucleotide edge. Nevertheless, a problem with side chain deprotection still exists. Literature presents examples of side chain protecting groups such as: Cys(S-t-Bu), Tyr(Trt), Ser(Trt), Cys(Trt), Lys(Boc), Ser(t-Bu), Arg(Pbf), Trp(Boc), His(Trt). These protecting groups, requiring cleavage by strong acids, trigger depurination and thus, the synthetic yield is reduced dramatically. It should be noted that in most cases reported in literature, the synthesis of the peptide-oligonucleotide conjugates was performed using amino acids with no functional groups at their side chain.

In fragmental conjugation (segmental condensation), peptide-oligonucleotide conjugates are synthesized through various linkers such as: (A) 2-amino ribose linker;⁸⁰ (B) maleimide linker;^{44,47,64,81} (C) isocyanate to form urea derivative;s⁸² (D) amide bond via formation of a thioester intermediate;⁸³ (E) thio ether formation;⁶⁶ (F) disulfide bond formation;^{41,84,85} (G) hydrazone formation from aldehyde and hydrazine;⁸⁶ and (H) aldehyde to form a linkage via thiazolidine, oxime and hydrazine bridge.⁸⁷

Like sequential synthesis, fragmental synthesis of POCs according to current methods has several limitations. Specifically, the two constituents (ODN and peptide) may have different solubility properties that can reduce considerably the yield of the formed hybrid. In addition, for conjugation, the two fragments must be well purified and thus there is a significant loss of starting material and of conjugate. In some cases, pre-modification, either in solution or on the solid support, is required. This may add some difficulties in the synthetic strategy. In addition, since the conjugation reaction takes place in solution, one of the fragments must be used in excess and can't be recovered and recycled. Another problem in this approach is related to possible folding of the two components resulting in the formation of an uncreative species. Finally, due to the functional side chains of the peptide, the range of an appropriate modified binding site is limited.

There is an urgent need in the art to develop a general synthetic procedure for preparing peptide-oligonucleotide conjugates that permits the start of the synthesis either from the peptide or from the oligonucleotide side, that can be conducted under mild conditions, that can be used to synthesize alternating sequences, and that is applicable to the synthesis of a wide variety of peptide-oligonucleotide conjugates constructed from alternate peptide and oligonucleotide blocks.

### SUMMARY OF THE INVENTION

The present invention provides new reagents and methods for the synthesis of peptide-oligonucleotide conjugates (POC), which include the use of appropriate protecting groups for the amino acid (AA) α-amino site and side chains that can be cleaved under mild conditions, and which further include the use of appropriate reagents for peptide-oligonucleotide coupling. The methods of the present invention can be conducted under mild conditions on solid support, can be performed manually or by a synthesizer, can be used to synthesize any peptide-oligonucleotide conjugates, including conjugates comprising alternating peptide-oligonucleotide sequences, and are applicable to the synthesis of a wide variety of peptide-oligonucleotide conjugates constructed from peptide and oligonucleotide blocks.

The present invention relates to a method for the preparation of a peptide-oligonucleotide conjugate (POC), by performing at least one coupling between an α-amino protected amino acid and a nucleotide so as to form a peptide-oligonucleotide conjugate having at least one amino acid-nucleotide bond. The assembly of the POC is conducted using one or more coupling reagents compatible with peptide synthesis, as defined herein. Furthermore, where appropriate, the amino acid and/or nucleotide may further comprise additional protecting groups that are orthogonal to (i.e., compatible with) the α-amino protecting group. The α-amino protecting group is removed prior to each amino acid-amino acid coupling step using a deprotecting agent that is compatible with any one or more protecting groups present in the oligonucleotide-peptide conjugate.

As contemplated herein, the applicants of the present invention have developed new methodology of peptide synthesis that is compatible with the synthesis of POC, under mild neutral conditions on solid support. A) New peptide building blocks were prepared. B) An o-nitrophenyl sulphenyl group (Nps) was used for α-amino protection. C) New mild conditions for removal of the Nps group (thioacetamide/dichloroacetic acid) were discovered. D) Protecting units for AA's side-chains were identified and selected, which are orthogonal to (compatible with) the Nps-group (e.g. (R)₄Si, BnSyl, Fmoc and Fm). In particular, it was shown that Fmoc and Fm side-chain protecting units are stable in acidic media and can be easily removed by fluoride anion under neutral conditions. E) Use of the new combination of Nps and Fmoc/Fm protecting groups permitted the synthesis of desired peptides in good yield and satisfactory purity. F) Different coupling reagents (e.g., HBTU, BOP, DCC, HATU, HDTU, PDOP) were tested in peptide synthesis. G) Oligonucleotides were synthesized by a combination of coupling reagents developed in peptide synthesis and the hydrogen phosphonate approach for phosphate bond formation. Particularly, it was also found that the combination of H-phosphonate approach using coupling reagents (e.g., HDTU, HATU, BOP-Cl, BrOP, ClOP, PyBrop, PyClop organophosphorochloridates) provides an effective method for ODN synthesis, which is compatible with the synthesis of peptides.

A new method of peptide-oligonucleotide conjugate synthesis under mild conditions on solid support was thus developed. This method can be performed manually or by a synthesizer and can be applied for the synthesis of various peptide-oligonucleotide conjugates, especially base or acid sensitive, constructed from alternate peptide and oligonucleotide blocks, branched or cyclic.

According to one embodiment, the present invention relates to a method for the preparation of a peptide-oligonucleotide conjugate (POC), comprising the step of performing at least one coupling between an α-amino protected amino acid and a nucleotide so as to form a peptide-oligonucleotide conjugate having at least one amino acid-nucleotide bond; wherein the amino acid or nucleotide further comprise one or more orthogonal protecting groups where required; wherein each coupling step is conducted in the presence of a coupling reagent compatible with peptide synthesis; and wherein the α-amino protecting group is removed prior to each amino acid-amino acid coupling step using a deprotecing agent compatible with any one or more protecting groups present in the oligonucleotide-peptide conjugate. In one currently preferred embodiment, the α-amino protecting group is N-α-o-nitrophenyl sulphenyl (N-α-Nps). In another embodiment, the α-amino protecting group is p-azidobenzyloxycarbonyl (ACBZ).

In another embodiment, the present invention relates to a method for the preparation of a peptide-oligonucleotide conjugate (POC), comprising the step of performing at least one coupling between an N-α-o-nitrophenyl sulphenyl (N-α-Nps) protected amino acid and a nucleotide so as to form a peptide-oligonucleotide conjugate having at least one amino acid-nucleotide bond; wherein the N-α-Nps protected amino acid or nucleotide further comprise one or more orthogonal protecting groups where required; wherein each coupling step is conducted in the presence of a coupling reagent compatible with peptide synthesis; and wherein the N-α-Nps protecting group is removed prior to each amino acid-amino acid coupling step using a deprotecting agent compatible with any one or more protecting groups present in the oligonucleotide-peptide conjugate.

In yet another embodiment, the present invention relates to a method for the preparation of a peptide-oligonucleotide conjugate (POC), comprising the steps of (a) providing a first N-α-o-nitrophenyl sulphenyl (N-α-Nps)-protected amino acid or a first nucleotide; (b) coupling, in any order, at least a second N-α-Nps-protected amino acid and/or at least a second nucleotide to the first N-α-Nps-protected amino acid or the first nucleotide; and (c) repeating step (b) as necessary, so as to form a peptide-oligonucleotide conjugate having at least one amino acid-nucleotide bond; wherein each coupling step is conducted in the presence of a coupling reagent compatible with peptide synthesis; and wherein the N-α-Nps protecting group is removed prior to each amino acid-amino acid coupling step using thioacetamide in the presence of dichloroacetic acid.

A coupling reagent which is compatible with peptide synthesis is used in the synthesis of the POC. Examples of such coupling reagents include but are not limited to 1-hydroxybenzotriazole (HOBt), 3-hydroxy-3,4-dihydro-1,2,3-benzotriazine-4-one (HOoBt), N-hydroxysuccinimide (NHS), dicyclohexylcarbodiimide (DCC), diisopropylcarbodiimide (DIC), 1-ethyl-3-(3-dimethylaminopropyl)-carbodiimide (EDAC), 2-(1H-7-azabenztriazol-1-yl)-1,1,3,3-tetramethyluronium hexafluoro phosphate (HATU), 2-(1H-benzotriazol-1-yl)-1,1,3,3-tetramethyluronium hexafluorophosphate (HBTU), 3,4-dihydro-1,2,3-benzotriazin-4-one-3-oxy tetramethyluronium hexafluorophosphate (HDTU), benzotriazol-1-yloxytris(dimethylamino)phosphonium hexafluoro phosphate (BOP), benzotriazol-1-yloxytris-(pyrrolidino)-phosphonium hexafluoro phosphate (PyBop), (3,4-dihydro-1,2,3-benzotriazin-4-one-3-oxy)diethyl phosphate (DEPBt), 3,4-dihydro-1,2,3-benzotriazin-4-one-3-oxy tris-(pyrrolidino)-phosphonium hexafluorophosphate (PDOP), 2-(benzotriazol-1-yloxy)-1,3-dimethyl-2-pyrrolidin-1-yl-1,3,2-diazaphospholidinium hexafluorophosphonate (BOMP), 5-(1H-7-azabenzotriazol-1-yloxy)-3,4-dihydro-1-methyl 2H-pyrrolium hexachloroantimonate (AOMP), (1H-7-azabenzotriazol-1-yloxy)tris(dimethylamino) phosphonium hexafluoroposphate (AOP), 5-(1H-Benzotriazol-1-yl)-3,4-dihydro-1-methyl 2H-pyrrolium hexachloroantimonate: N-oxide (BDMP), 2-bromo-3-ethyl-4-methyl thiazolium tetrafluoroborate (BEMT), 2-bromo-1-ethyl pyridinium tetrafluoroborate (BEP), 2-bromo-1-ethyl pyridinium hexachloroantimonate (BEPH), N-(1H-benzotriazol-1-ylmethylene)-N-methylmethanaminium hexachloroantimonate N-oxide (BOMI), N,N′-bis(2-oxo-3-oxazolidinyl) phosphinic chloride (BOP-Cl), 1-(1H-benzotriazol-1-yloxy)phenylmethylene pyrrolidinium hexachloroantimonate (BPMP), 1,1,3,3-bis(tetramethylene) fluorouronium hexafluorophosphate (BTFFH), chloro(4-morphoino)methylene morpholinium hexafluorophosphate (CMMM), 2-chloro-1,3-dimethyl-1H-benzimidazolium hexafluorophosphate (CMBI), 2-fluoro-1-ethyl pyridinium tetrafluoroborate (FEP), 2-fluoro-1-ethyl pyridinium hexachloroantimonate (FEPH), 1-(1-pyrrolidinyl-1H-1,2,3-triazolo[4,5-b]pyridin-1-ylmethylene)pyrrolidinium hexafluorophosphate N-oxide (HAPyU), O-(1H-benzotriazol-1-yl)-N,N,N′,N;-bis(pentamethylene)uronium hexafluorophosphate (HBPipU), O-(1H-benzotriazol-1-yl)-N,N,N0,N0-bis(tetramethylene)urinium hexafluorophosphate (HBPyU), (1H-7-azabenzotriazol-1-yloxy)tris(pyrrolidino)phosphonium hexafluorophosphate (PyAOP), bromotripyrrolidinophosphonium hexafluorophosphate (PyBrOp), chlorotripyrrolidinophosphonium hexafluorophosphate (PyClOP), 1,1,3,3-bis(tetramethylene) chlorouronium hexafluorophosphate (PyClU), tetramethylfluoromamidinium hexafluorophosphate (TFFH), triphosgene, triazine-based reagents [cyanuric chloride, cyanuric fluoride, 4-(4,6-dimethoxy-1,3,5-triazin-2-yl)-4-methylmorpholinium chloride (DMT-MM), 2-chloro-4,6-dimethoxy-1,3,5-triazine (CDMT)], bis(2-chlorophenyl) phosphorochloridate, diphenyl phosphorochloridate, diphenyl phosphoroazide (DPPA) and any combination thereof.

A currently preferred coupling reagent is 2-(1H-7-azabenztriazol-1-yl)-1,1,3,3-tetramethyluronium hexafluoro phosphate (HATU). Another currently preferred coupling reagent is 3,4-dihydro-1,2,3-benzotriazin-4-one-3-oxy tetramethyluronium hexafluorophosphate (HDTU). Another currently preferred coupling reagent is N,N′-bis(2-oxo-3-oxazolidinyl) phosphinic chloride (BOP-Cl) Another currently preferred coupling reagent is an organophosphoro halogenate or a pseudohalogenate such as diphenyl phosphorochloridate and diphenylphosphoroazide (DPPA). Another currently preferred coupling reagent is a halogeno tris(organo)phosphonium hexafluoro phosphate such as bromo tris(dimethylamino)phosphonium hexafluoro phosphate (BrOP), chlorotris(dimethylamino)phosphonium hexafluoro phosphate (ClOP), bromotripyrrolidinophosphonium hexafluorophosphate (PyBrOp) and chlorotripyrrolidinophosphonium hexafluorophosphate (PyClOP).

The amino acid used in the methods of the present invention can be any natural or unnatural amino acid, including but not limited to glycine, alanine, valine, leucine, isoleucine, proline, arginine, lysine, histidine, serine, threonine, aspartic acid, glutamic acid, asparagine, glutamine, cysteine, homocysteine, cystine, methionine, ornithine, norleucine, phenylalanine, tyrosine, tryptophan, beta-alanine, homoserine, homoarginine, isoglutamine, pyroglutamic acid, gamma-aminobutryic acid, citrulline, sarcosine, and statine. Preferably the amino acid is protected with a N-α-Nps protecting group.

In addition, one or more of the amino acids used in the methods of the present invention can contain a side chain that requires protection during the synthesis. Examples of such amino acids include but are not limited to arginine, lysine, aspartic acid, asparagine, glutamic acid, glutamine, histidine, cysteine, homocysteine, ornithine, serine, homoserine, threonine, homoarginine, citrulline and tyrosine.

Suitable protecting groups are groups that can be removed under mild conditions, such as a silyl protecting group, which can be removed by reaction with fluoride. Applicants have discovered that suitable silyl protecting groups are groups of the formula (R)₄Si wherein each R is independently of the other an unsubstituted or substituted alkyl, alkylaryl, aryl, oxyalkyl, oxyalkylaryl, or oxyaryl.

A currently preferred silyl protecting group is a silanoxylbenzylcarbonyl protecting group represented by the structure:

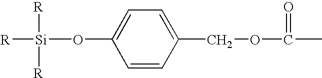

wherein each R is independently of the other selected from the group consisting of an unsubstituted or substituted alkyl, alkylaryl, aryl, oxyalkyl, oxyalkylaryl and oxyaryl.

In accordance with this embodiment, the protected amino acid is represented by the following structure of formula (I):

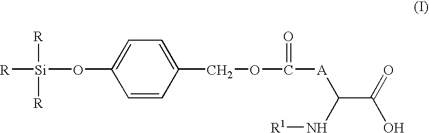

wherein
   A represents a side chain residue of the amino acid;
   R is independently selected from the group consisting of an unsubstituted or substituted alkyl, alkylaryl, aryl, oxyalkyl, oxyalkylaryl and oxyaryl; and
   R¹ represents hydrogen or an amino protecting group.

A currently preferred protecting group for the alpha-amino group of the compound of formula (I) is nitrophenyl sulphenyl (Nps), i.e. a compound of formula (I) wherein R¹ is Nps. In accordance with this preferred embodiment, the side-chain protected amino acid is represented by the formula (II):

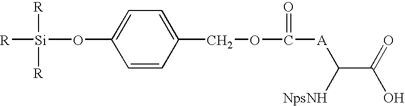

### (II)

In one embodiment, the novel side chain protecting group is introduced via a 4-nitrophenyl silanoxybenzyl carbonate of the formula (III):

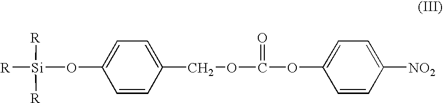

The present invention also provides a method for preparing a side-chain protected amino acid of formula (I):

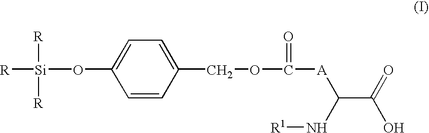

wherein
   A represents a side chain residue of the amino acid;
   R is independently selected from the group consisting of an unsubstituted or substituted alkyl, alkylaryl, aryl, oxyalkyl, oxyalkylaryl and oxyaryl; and
   R¹ represents hydrogen or an amino protecting group.

The method comprises the step of reacting the amino acid with a compound of the formula (III):

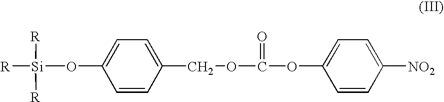

thereby forming the side-chain protected amino acid.

The present invention also encompasses novel 4-nitrophenyl ester silanoxybenzyl esters of formula (III), and their use in protecting side chain groups of amino acids.

In a particular embodiment, the silyl protecting group is represented by the structure:

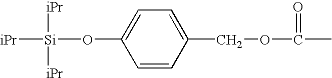

In accordance with this embodiment, the protected amino acid is represented by the following structure (IV):

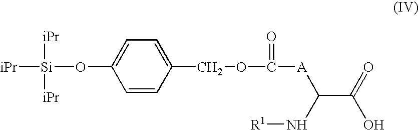

wherein A and R¹ are as defined above.

Furthermore, In accordance with this embodiment, the novel side chain protecting group is introduced via a 4-nitrophenyl-4-triisopropylsilanoxybenzyl (BnSyl) carbonate (V):

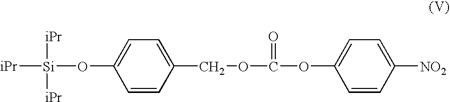

The present invention also encompasses a 4-nitrophenyl silanoxybenzyl carbonate of formula (V), and their use in protecting side chain groups of amino acids.

In general, a reagent for protection of side chains can be presented by formula

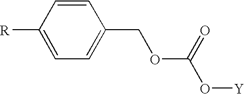

wherein R is a group which is suitable to cascade decomposition of a substituted benzyloxycarbonyl function (e.g. a silyl group), and Y is a leaving group selected from the group consisting of: p-nitrophenyl, pentafluorophenyl, trichlorophenyl, 3-3,4-dihydro-1,2,3-benzotriazin-4-one, N-succinimide, N-benzotriazole, N-azobenzotriazole and analogous derivatives, widely used in peptide chemistry for preparation of active esters.

The removal of such a protecting group is represented schematically in scheme 1, for example when R═(R′)₃SiO.

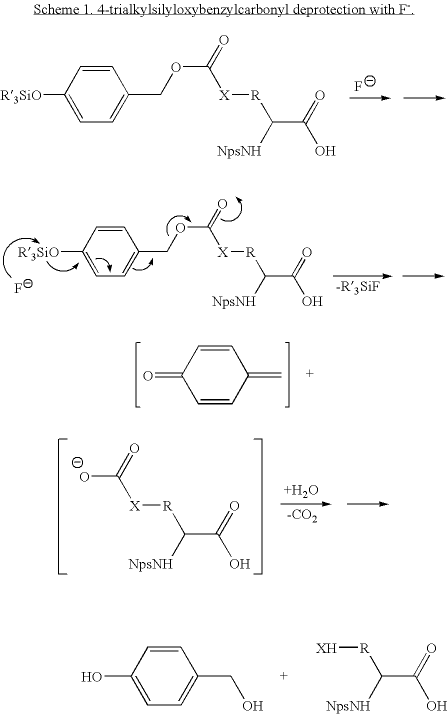

The removal of such a protecting group is represented schematically in scheme 2, for example when R═N₃ (ACBZ group).

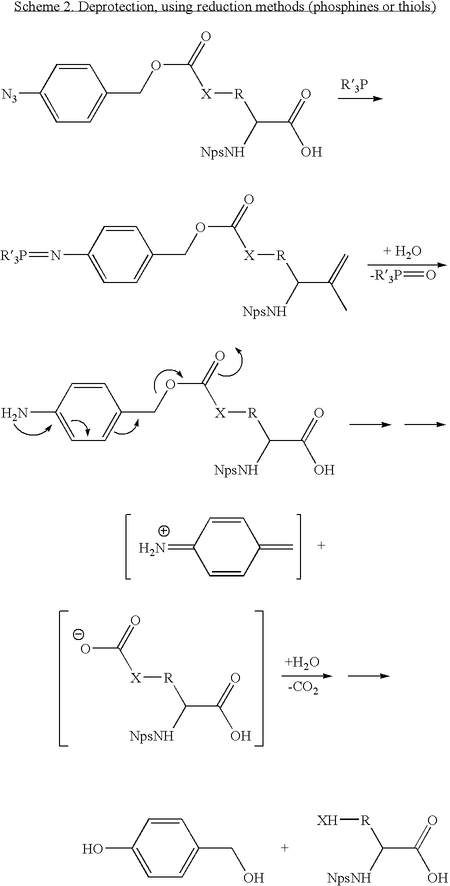

Other suitable protecting groups include N^{α}-9-fluorenylmethoxycarbonyl (Fmoc) and N^{α}-9-fluorenylmethyl (Fm) derivatives.

The synthesis of the oligonucleotide is conducted by any known oligonucleotide synthetic approach, including a phosphate approach, an H-phosphonate approach, or a phosphite approach. A currently preferred method is the H-phosphonate method.

The methods of the present invention can be carried out in solution phase or on a solid support. In addition, the synthesis can be conducted in any order, such that the synthesis can begin with the oligonucleotide followed by synthesis of the peptide, or vice versa. In addition, segments of the peptide or oligonucleotide can be synthesized, followed by segments of the other building block, and this can be repeated in an alternating mode, thereby producing alternate peptide-oligonucleotide sequences.

The present invention thus overcomes the problems of prior art POC synthesis, and provides a general synthetic procedure for preparing peptide-oligonucleotide conjugates that is applicable to the synthesis of a wide variety of peptide-oligonucleotide conjugates.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: NMR spectra of NPS-Leu

FIG. 2: MS-ES of penta-peptides synthesized by NPS method

### DETAILED DESCRIPTION OF THE PRESENT INVENTION

The present invention provides new reagents and methods for the synthesis of peptide-oligonucleotide conjugates (POC), which include the use of appropriate protecting groups for the amino acid (AA) α-amino site and the side chains that can be cleaved under mild conditions, and which further include the use of appropriate reagents for peptide-oligonucleotide coupling. The methods of the present invention can be conducted under mild conditions on solid support, can be performed manually or by a synthesizer, can be used to synthesize alternating peptide-oligonucleotide sequences, and are applicable to the synthesis of a wide variety of peptide-oligonucleotide conjugates constructed from alternate peptide and oligonucleotide blocks, which can be branched or cyclic. Accordingly, in one embodiment, the present invention relates to a method for the preparation of a peptide-oligonucleotide conjugate (POC), comprising the steps of (a) providing a first N-α-o-nitrophenyl sulphenyl (N-α-Nps)-protected amino acid or a first nucleotide; (b) coupling, in any order, at least a second N-α-Nps-protected amino acid and/or at least a second nucleotide to the first N-α-Nps-protected amino acid or the first nucleotide; and (c) repeating step (b) as necessary, so as to form a peptide-oligonucleotide conjugate having at least one amino acid-nucleotide bond; wherein each coupling step is conducted in the presence of a coupling reagent compatible with peptide synthesis; and wherein the N-α-Nps protecting group is removed prior to each amino acid-amino acid coupling step using thioacetamide in the presence of dichloroacetic acid.

In another embodiment, the present invention relates to a method for the preparation of a peptide-oligonucleotide conjugate (POC), comprising the steps of: (a) providing a first amino acid or a first nucleotide, wherein the first amino acid is a N-α-o-nitrophenyl sulphenyl (N-α-Nps)-protected amino acid; (b) coupling at least a second N-α-Nps-protected amino acid to the first amino acid or first oligonucleotide using a coupling reagent compatible with peptide synthesis; (c) coupling at least a second nucleotide to the first amino acid or first nucleotide using a coupling reagent compatible with peptide synthesis; wherein steps (b) and (c) are performed in any order; and (d) repeating steps (b) and (c) as necessary in any order; wherein the N-α-Nps protecting group is removed prior to each peptide coupling step using thioacetamide in the presence of dichloroacetic acid; thereby preparing the peptide-oligonucleotide conjugate.

Peptide-Oligonucleotide Assembly:

There are two different approaches that are currently used to synthesize peptide-oligonucleotide conjugates, the sequential (or stepwise) synthesis and the fragmental conjugation (segmental condensation). In the sequential synthesis, the peptide and oligonucleotide are synthesized sequentially on automatic synthesizers.

Although it is contemplated that the methods of the present invention are conducted by a stepwise approach, it is apparent to a person skilled in the art that the methods of the present invention are also applicable to the synthesis of POCs by a fragmental approach. In fragmental conjugation, peptide-oligonucleotide conjugates are synthesized through various linkers such as: (A) 2-amino ribose linker;⁸⁰ (B) maleimide linker;^{44,47,64,81} (C) isocyanate to form urea derivatives;⁸² (D) amide bond via formation of thioester intermediate;⁸³ (E) thioether formation;⁶⁶ (F) disulfide bond formation;^{41,84,85} (G) hydrazone formation from aldehyde and hydrazinee;⁸⁶ (H) aldehyde to form a linkage via thiazolidine, oxime and hydrazine bridge.⁸⁷

It is apparent to a person skilled in the art, that in addition to the sequential and fragmental methods, the peptide-oligonucleotides can be synthesized by any other synthetic approach.

Peptide Synthesis:

The peptide segments of the present invention are prepared using amino acid (AA) building blocks, which can be any natural or unnatural amino acid, including but not limited to glycine, alanine, valine, leucine, isoleucine, proline, arginine, lysine, histidine, serine, threonine, aspartic acid, glutamic acid, asparagine, glutamine, cysteine, homocysteine, cystine, methionine, ornithine, norleucine, phenylalanine, tyrosine, tryptophan, beta-alanine, homoserine, homoarginine, isoglutamine, pyroglutamic acid, gamma-aminobutryic acid, citrulline, sarcosine, and statine.

α-amino Protecting Groups:

For protection of the α-amino group of the AA, any group which is resistant to fluoride anion, but cleaved under mild neutral or slightly acidic conditions, can be used, including but not limited to: Nps (o-nitrophenyl sulphenyl), o- and p-nitrobenzenosulfonyl (o- and pNBS), dinitrobenzenosulfonyl (dNBS), benzothiazole-2-sulfonyl (Bts), dithiasuccinoyl (Dts), and Alloc groups.

In one embodiment, introduction of the Nps α-amino protecting group is achieved by reacting the free amino group acid with o-nitrophenyl sulphenyl chloride as outlined in Scheme 3.

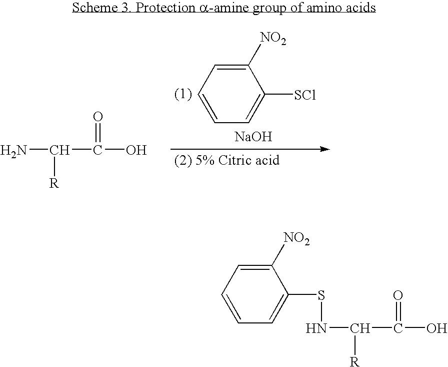

Removal of this protecting group can be achieved by using thio-containing reagents in the presence of acetic acid or its derivatives, for example, by using thioacetamide with a catalytic amount of acetic acid in methanol, thiourea or sodium thiosulphate in the same conditions, 2-mercaptopyridine in DMF or methylene chloride with a catalytic amount of acetic acid. As demonstrated herein, it was found that the Nps-group can be cleaved by reaction with thioacetamide with a catalytic amount of dichloroacetic acid. The applicants of the present invention have surprisingly and unexpectedly found these conditions to be so mild that all other protecting groups are unaffected.

In addition, in the absence of protected cysteine residues, the Nps-group can be removed by thiols or phosphines in regular manner used in synthesizing peptides.

### Side Chain Protecting Groups:

One or more of the amino acids used in the methods of the present invention can contain a side chain that needs to be protected during the synthesis. Examples of such amino acids are arginine, lysine, aspartic acid, asparagine, glutamic acid, glutamine, histidine, cysteine, homocysteine, hydroxyproline, ornithine, serine, homoserine, threonine, tryptophan, homoarginine, citrulline and tyrosine.

Suitable protecting groups are groups that can be removed by mild conditions, such as a silyl protecting group, which can be removed by reaction with fluoride anion. Applicants have discovered that suitable silyl protecting groups are groups of the formula (R)₄Si wherein each R is independently of the other an unsubstituted or substituted alkyl, alkylaryl, aryl, oxyalkyl, oxyalkylaryl, or oxyaryl.

The term “alkyl” as used herein alone or as part of another group refers to both straight and branched chain hydrocarbons, containing 1 to 20 carbons, preferably 1 to 10 carbons, more preferably 1 to 8 carbons, such as methyl, ethyl, propyl, isopropyl, butyl, t-butyl, isobutyl, pentyl, hexyl, isohexyl, heptyl, octyl, nonyl, decyl, undecyl, dodecyl and the like and, the various branched chain isomers thereof. Where alkyl groups as defined above have single bonds for attachment to other groups at two different carbon atoms, they are termed “alkylene” groups. The alkyl group can be unsubstituted or substituted through available atoms by one or more of the groups selected from halo such as F, Br, Cl or I, haloalkyl such as CF₃, alkyl, alkoxy, haloalkoxy, trifluoromethoxy, alkenyl, alkynyl, cycloalkyl, cycloalkylalkyl, cycloheteroalkyl, cycloheteroalkylalkyl, cycloalkenyl, cycloalkenylalkyl, cycloalkynyl, cycloalkynylalkyl, aryl, heteroaryl, arylalkyl, aryloxy., aryloxyalkyl, aryloxyaryl, arylalkyloxy, arylalkenyl, arylalkynyl, arylazo, heteroarylalkyl, heteroarylalkenyl, heteroarylheteroaryl, heteroaryloxy, hydroxy, hydroxyalkyl, nitro, cyano, amino, alkanoyl, aroyl, alkylamino, dialkylamino, arylamino, diarylamino, thio, alkylthio, arylthio, arylalkylthio, heteroarylthio, alkoxyarylthio, acyl, alkylcarbonyl, arylcarbonyl, allyl-aminocarbonyl, arylaminocarbonyl, alkoxycarbonyl, aryloxycarbonyl, alkoxycarbonyloxy, aminocarbonyl, alkylaminocarbonyl, arylaminocarbonyl, alkylcarbonyloxy, arylcarbonyloxy, alkylamido, alkanoylamino, alkylcarbonylamino, arylcarbonylamino, sulfonyl, alkylsulfonyl, arylsulfonyl, aminosulfinyl, sulfonyl, alkylsulfinyl, arylsulfinyl, aminosulfinyl, arylsulfinylalkyl, arylsulfonylamino and aminocarbonyl.

The term “aryl” as used herein alone or as part of another group refers to an aromatic ring system containing from 6-10 ring carbon atoms and up to a total of 15 carbon atoms. The aryl ring can be a monocyclic, bicyclic, tricyclic and the like. Non-limiting examples of aryl groups are phenyl, naphthyl including 1-naphthyl and 2-naphthyl, and the like. The aryl group can optionally be substituted through available carbon atoms with one or more groups defined hereinabove for alkyl.

The term “alkylaryl” as used herein alone or as part of another group refers to an alkyl group as defined herein linked to an aryl group as defined herein.

The term “oxy” as used herein refers to the group “O”. The terms “oxyalkyl” “oxyalkylaryl”, or “oxyaryl” refer to an alkyl, alkylaryl or aryl, respectively, that are bound through an oxygen atom.

A currently preferred silyl protecting group is a silanoxylbenzylcarbonyl protecting group represented by the structure:

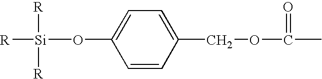

wherein each R is independently of the other selected from the group consisting of an unsubstituted or substituted alkyl, alkylaryl, aryl, oxyalkyl, oxyalkylaryl and oxyaryl.

In accordance with this embodiment, the protected amino acid is represented by the following structure of formula (I):

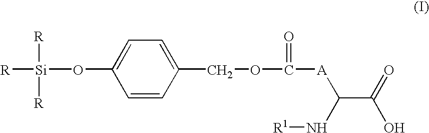

wherein
   A represents a side chain residue of the amino acid;
   R is independently selected from the group consisting of an unsubstituted or substituted alkyl, alkylaryl, aryl, oxyalkyl, oxyalkylaryl and oxyaryl; and
   R¹ represents hydrogen or an amino protecting group.

The method comprises reacting the amino acid with a compound of the formula (III):

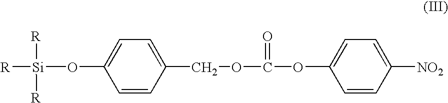

thereby forming the side-chain protected amino acid.

The present invention also encompasses 4-nitrophenyl silanoxybenzyl carbonates of formula (III), and their use in protecting side chain groups of amino acids.

In a particular embodiment, the silyl protecting group is represented by the structure:

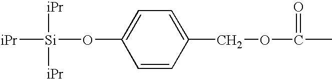

In accordance with this embodiment, the protected amino acid is represented by the following structure of formula (IV):

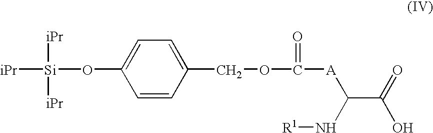

wherein A and R¹ are as defined above.

Furthermore, in accordance with this embodiment, the novel side chain protecting group (BnSyl) is introduced via a 4-nitrophenyl-4-triisopropylsilanoxybenzyl carbonate (V).

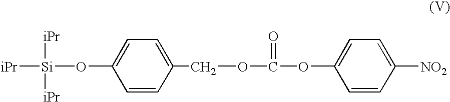

R is independently selected from the group consisting of an unsubstituted or substituted alkyl, alkylaryl, aryl, oxyalkyl, oxyalkylaryl and oxyaryl; and
R¹ represents hydrogen or an amino protecting group.

A currently preferred protecting group for the alpha-amino group of the compound of formula (I) is nitrophenyl sulphenyl (Nps), i.e. a compound of formula (I) wherein R¹ is Nps. In accordance with this preferred embodiment, the side-chain protected amino acid is represented by the formula (II):

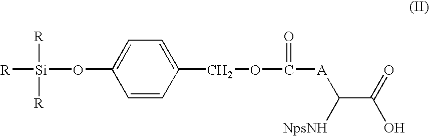

The novel side chain protecting group can be introduced via a 4-nitrophenyl silanoxybenzyl carbonate of the formula (III):

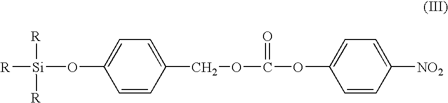

The present invention also provides a method for preparing a side-chain protected amino acid of formula (I):

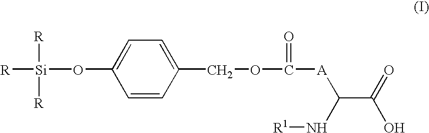

wherein
A represents a side chain residue of the amino acid;

The present invention also encompasses 4-nitrophenyl silanoxybenzyl carbonates of formula (V), and their use in protecting side chain groups of amino acids.

Not wishing to be bound to any particular mechanism or theory, it is contemplated that the attack of fluoride anion on silicon will cause the cascade decomposition according to scheme 1.

Other suitable protecting groups include N^{α}-9-fluorenylmethoxycarbonyl (Fmoc) and N^{α}-9-fluorenylmethyl (Fm) derivatives.

The selection of groups for side chain protection was performed in accordance to compatibility with Nps-strategy (Table 1):

TABLE 1

| Amino acid | Protecting Group for Side Chain |
|---|---|
| Gln | Fmoc |
| Thr | SiR₃, Alloc, BnSyl, Fmoc, Fm, |
| Asn | Fmoc |
| Ser | SiR₃, Alloc, BnSyl, Fmoc, Fm, |
| Tyr | SiR₃, Alloc, BnSyl, Fmoc, Fm, |
| Lys | BnSyl, Fmoc, Alloc\grave{ } |
| Trp | Fmoc, Alloc, BnSyl, Dnp |
| Arg | Fmoc_{2,} Alloc, Alloc₂, BnSyl, BnSyl₂, |
| | ACBZ₃, (ACBZ)₂, Teoc, Teoc₂, |
| Asp | Fm, All, Pac, Tce, Nbn, |
| His | Alloc, Fmoc, BnSyl, Tos, Dnp |
| Orn | BnSyl, Fmoc, Alloc\grave{ } |
| Cys | Fm, Alloc |
| Hse | SiR₃, Alloc, BnSyl, Fmoc, Fm, |
| Hyp | SiR₃, Alloc, BnSyl, Fmoc, Fm, |
| Glu | Fm, All, Pac, Tce, Nbn, |

For example, arginine can be used without protection or it can be protected by groups including but not limited to: Fmoc, BnSyl, 2-(trimethylsilyl)ethoxycarbonyl (Teoc), 2-(trimethylsilyl)ethylsulphonyl (SES) groups.

Nps-strategy is particularly advantageous for use in solid phase peptide synthesis. For solution methods of peptide synthesis the applicants have developed another combination of α-amino and side chain protecting groups, using ACBZ (p-azidobenzyloxycarbonyl) residue for protection of the α-amino group of the AA, and different groups for side chains protection as specified in Table 2.

TABLE 2

| Amino acid | Protecting Group for Side Chain |
|---|---|
| Gln | Fmoc |
| Thr | SiR₃, Alloc, BnSyl, Fmoc, Fm, |
| Asn | Fmoc |
| Ser | SiR₃, Alloc, BnSyl, Fmoc, Fm, |
| Tyr | SiR₃, Alloc, BnSyl, Fmoc, Fm, |
| Lys | BnSyl, Fmoc, Alloc\grave{ } |
| Trp | Fmoc, Alloc, BnSyl, Dnp |
| Arg | Fmoc_{2,} Alloc, Alloc₂, BnSyl, BnSyl₂, |
| | Teoc, Teoc₂, |
| Asp | Fm, All, Pac, Tce, Nbn, |
| His | Alloc, Fmoc, BnSyl, Tos, Dnp |
| Orn | BnSyl, Fmoc, Alloc\grave{ } |
| Cys | Fm, Alloc |
| Hse | SiR₃, Alloc, BnSyl, Fmoc, Fm, |
| Hyp | SiR₃, Alloc, BnSyl, Fmoc, Fm, |
| Glu | Fm, All, Pac, Tce, Nbn, |

The ACBZ α-amino protecting group is represented by the structure:

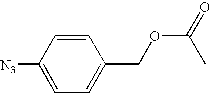

The ACBZ α-amino protected amino acid is thus represented by the following structure of formula (VI):

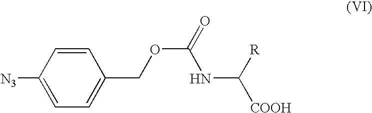

wherein R represents a side chain residue of an amino acid.

In one embodiment, introduction of the ACBZ α-amino protecting group is achieved by reacting the free amino group acid with p-azidobenzyl chloroformate or the corresponding p-azidobenzyl carbonates as outlined in Scheme 4.

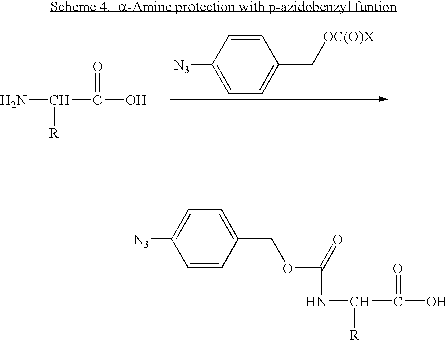

X=Cl, p-nitrophenyl, pentafluorophenyl, N-oxysuccinimide

The ACBZ protecting group is introduced, in one embodiment, via the carbonate of the formula (VII):

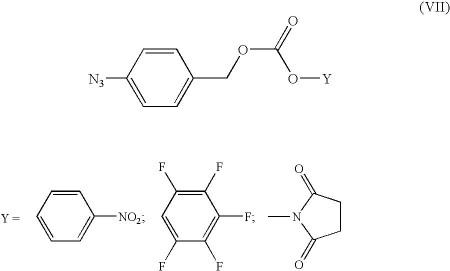

Removal of this protecting group can be achieved by using thio-containing reagents such as DTT or by using phosphines, followed by addition of water for phosphinimides hydrolysis and regeneration of the α-amino group.

Not wishing to be bound to any particular mechanism or theory, it is contemplated that the removal of ACBZ protecting group is achieved similar to mechanism presented in scheme 2.

### Side Chain Protecting Groups:

One or more of the amino acids used in the methods of the present invention can contain a side chain that requires protection during the synthesis. Examples of such amino acids are arginine, lysine, aspartic acid, asparagine, glutamic acid, glutamine, histidine, cysteine, homocysteine, hydroxyproline, ornithine, serine, homoserine, threonine, tryptophan, homoarginine, citrulline and tyrosine.

Suitable protecting groups are groups that can be removed under mild conditions. Preferred protecting group are 9-fluorenylmethyl-based protecting groups (Fmoc or Fm), which can be removed by reaction with fluoride anion.

It was shown by the applicants that the combination of ACBZ for α-amino group protection and Fmoc/Fm for side chain protection of amino acids is most suitable for peptide synthesis in solution, using stepwise or segment condensation methods, as further detailed in the experimental section.

### Solid Support:

Although it is possible to carry out the methods of the present invention is solution, it is contemplated that the methods of the present invention are conducted in the solid phase, on a solid resin or support.

The first synthetic strategy of solid-phase peptide synthesis (SPPS) was developed by R. B. Merriefeld in 1963.⁸⁸ Along with the development of related technologies such as reversed-phase high performance liquid chromatography (RP-HPLC) and mass spectrometry, the solid-phase method became a major technique in peptide synthesis.

The most commonly used resins for Boc solid-phase method are provided below. The hydroxymethylphenylacetamidomethyl resin (Pam resin) (a)^{89,90} is used for preparation of terminal free acids. The 4-methylbenzhydrylamine resin (MBHA resin) (b)⁹¹ is used for the preparation of terminal amide groups. Peptides, synthesized on these two resins, are cleaved from the resins by treatment with a strong acid such as anhydrous hydrogen fluoride (HF),⁹² trifluoromethanesulfonic acid (TMSA),⁹³ and trimethylsilyl trifluoromethanesulfonate.⁹³ The p-Nitrobenzophenone oxime resin (c) is used for the preparation of peptides holding their side protecting groups. Cleavage from this resin is implemented by nucleophiles such as N-hydroxypiperidine.⁹⁴ Peptides prepared on resin (d), bearing a 3-nitro-4-hydroxymethylbenzoyl group, are photocleavable by irradiation at 350 nm light.⁹⁵ Peptides synthesized on the (4-bromocrotonyl)aminomethyl resin (e) are cleaved by Pd(0)/morpholine treatment.⁹⁶

The most commonly used resins for F-moc solid-phase method are provided below. Cleavage from the hydroxymethylphenoxymethyl resin (Wang resin) (a)¹⁰⁰ and cleavage of side chains protecting groups is carried out by using TFA. The 2-chlorotrityl chloride resin (Trt-(2-Cl)resin) (b)¹⁰¹ enables cleavage from the resin of intact protected peptide. 4-(α-amino-2′,4′-dimethoxybenzyl)phenoxymethyl resin (c)¹⁰² is used for the formation of terminal amide.

Fluoride Anion Cleavable Linkers:

In order to retain the acid and/or base-sensitive substituents, mildly or neutrally cleavable linkers have also been developed. Among the latter, silyl linkers are of great promise because of their orthogonally cleavable property by fluoridolysis [Linkers and Cleavage Strategies in Solid-Phase Organic Synthesis and Combinatorial Chemistry. F. Guillier, D. Orain, M. Bradley. Chem. Rev. 2000, v. 100, p. 2091-2157].

Representative examples of silyl linkers are presented below:

### A)

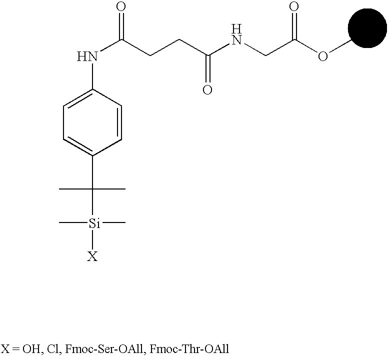

K. Nakamura e.a. Tetrahedron Lett., 1999, v.40, p. 515; Tetrahedron, 1999, v.55, p. 11253; Biosci., Biotechnol., Biochem., 2002, v.66, p. 225; Tetrahedron, 2000, v. 56, p. 6235
B) Benzyloxy(diisopropyl)silyl linker:

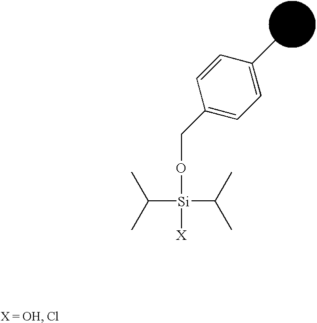

Akio Kobori, Kenichi Miyata, Masatoshi Ushioda, Kohji Seio, and Mitsuo Sekine. J. Org. Chem. 2002, v. 67, p. 476; Chem. Lett., 2002, p. 16.
C) Silyl linker for reverse-direction solid-phase peptide synthesis

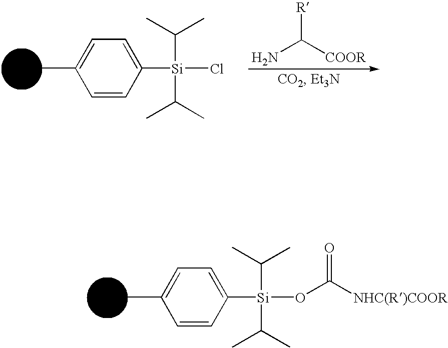

### B. H. Lipshutz and Y-J. Shin, Tetrahedron Lett., 2001, v. 42, p. 5629

D) (4-Methoxyphenyl)diisopropylsilylpropyl polystyrene

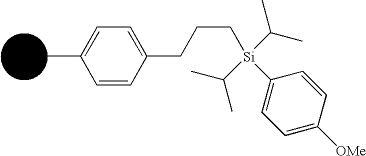

Yun Liao, Reza Fathi, and Zhen Yang. Journal of Combinatorial Chemistry, 2003, Vol. 5, No. 2, p. 79.
E) Pbs handle [D. G. Mullen, G. Barany. J. Org. Chem., 1988, v.53, p. 5240]:

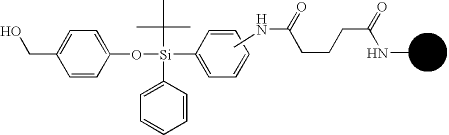

F) (2-Phenyl-2-trimethylsilyl)ethyl-(PTMSEL)-Linker [M. Wagner, S. Dziadek, and H. Kunz. Chem. Eur. J. 2003, v. 9, p. 6018]

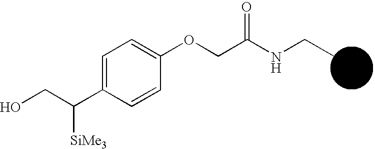

The main disadvantage of using these compounds lies in the complicated procedures for their preparation. For example, Pbs handle was prepared in 13 stages, and the PTMSEL linker was obtained in 7 stages, which limits their application in solid-phase chemistry.

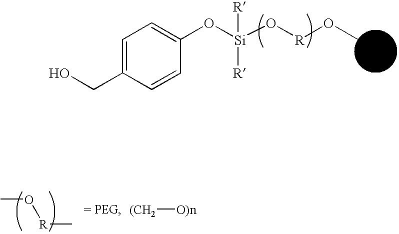

Currently preferred linkers are

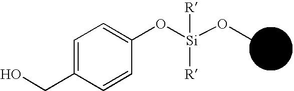

wherein R′ represents an alkyl or aryl group.

In a particular embodiment, the R′ group is Ph, i-Pr, t-Bu.

This novel linker can be prepared by a three-stage synthesis on the base of Merrifield (chloromethyl- or hydroxymethylstyrene copolymer) resin with direct loading of monomers (protected amino acids or oligonucleotides):

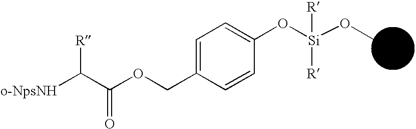

After modification, this linker can be also used for reverse-direction solid-phase synthesis:

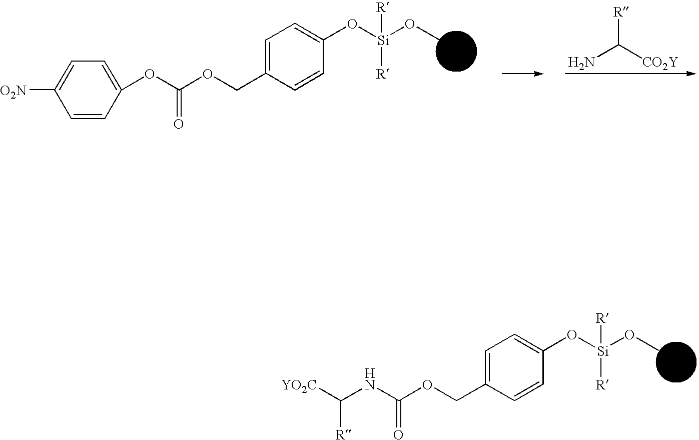

wherein Y is a protecting group

The high thermodynamic affinity of fluorine for silicon allows mild deprotection conditions using fluorine sources such as LiBF₄, KBF₄, KF, CsF, HBF₄, HF, PhCH₂NMe₃F (BTAF), tetrabutylammonium fluoride (TBAF), among them TBAF or HF/pyridine in THF or CsF in DMF/water or HF in acetonitrile are preferred methods for removal of biopolymers from solid support, as exemplified in Scheme 5:

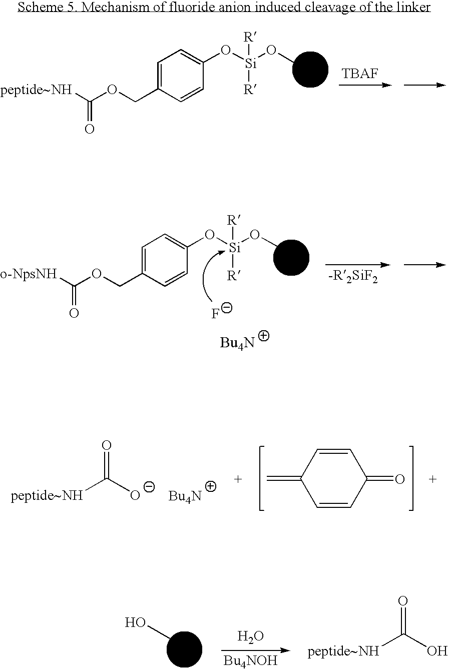

Excess of fluoride anion can be scavenged using methoxytrimethylsilane, leading to volatile trimethylsilyl fluoride and methanol.

The additional type of silicon-base resin, discovered by the applicants, is based on commercial available allyldimethylsilyl polystyrene (NovaBiochem). After modification (Scheme 6), this resin can be used for direct or reverse-type biopolymer synthesis:

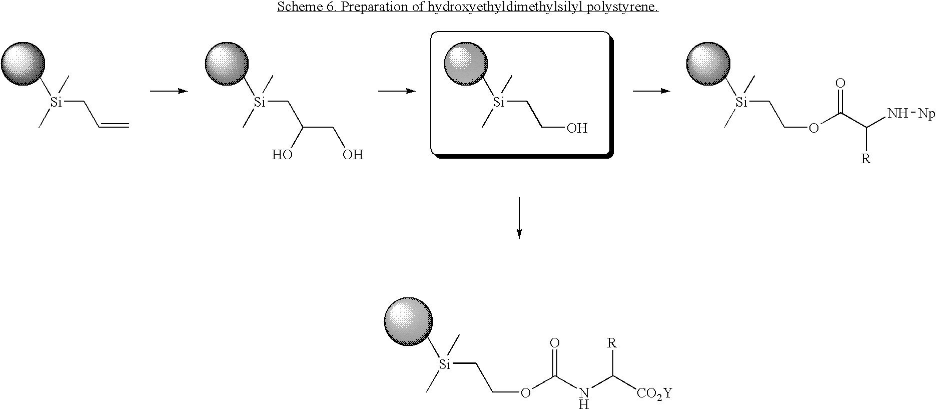

Taking into account the ability of the Fmoc-group to be removed by fluoride anion, the applicants have discovered that Fm-based linker can also been employed to release biopolymers from solid supports. This is the first example of non-silicon linker cleaved by fluoride anion. The preparation of this linker is exemplified in scheme 7:

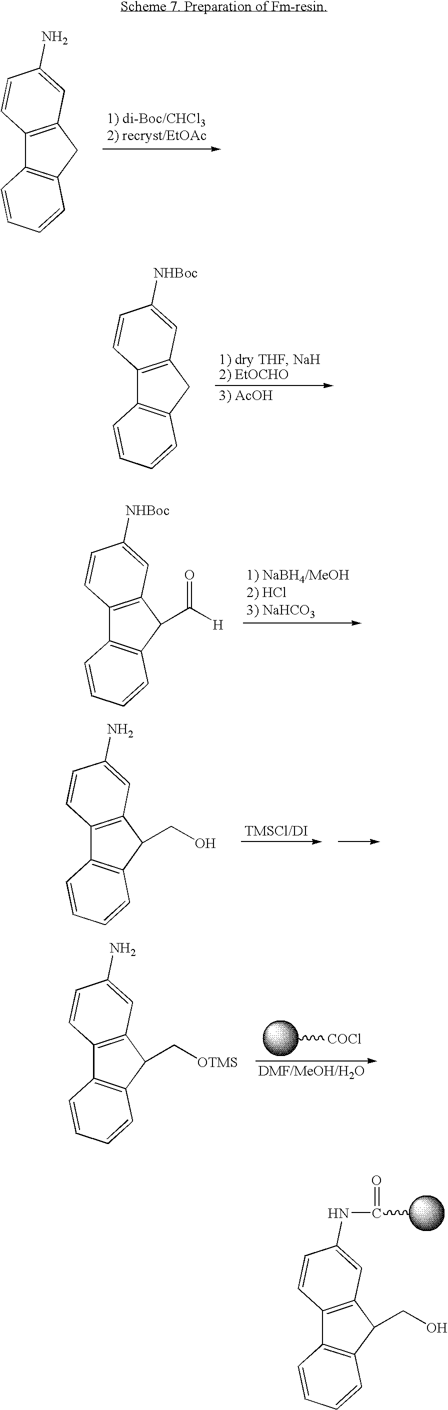

### Coupling Reagents

A coupling reagent which is compatible with peptide synthesis is used in the synthesis of the POC. Examples of such coupling reagents include but are not limited to 1-hydroxybenzotriazole (HOBt), 3-hydroxy-3,4-dihydro-1,2,3-benzotriazine-4-one (HOoBt), N-hydroxysuccinimide (NHS), dicyclohexylcarbodiimide (DCC), diisopropylcarbodiimide (DIC), 1-ethyl-3-(3-dimethylaminopropyl)-carbodiimide (EDAC), 2-(1H-7-azabenztriazol-1-yl)-1,1,3,3-tetramethyluronium hexafluoro phosphate (HATU), 2-(1H-benzotriazol-1-yl)-1,1,3,3-tetramethyluronium hexafluorophosphate (HBTU), 3,4-dihydro-1,2,3-benzotriazin-4-one-3-oxy tetramethyluronium hexafluorophosphate (HDTU), benzotriazol-1-yloxytris(dimethylamino)phosphonium hexafluoro phosphate (BOP), benzotriazol-1-yloxytris-(pyrrolidino)-phosphonium hexafluoro phosphate (PyBop), (3,4-dihydro-1,2,3-benzotriazin-4-one-3-oxy)diethyl phosphate (DEPBt), 3,4-dihydro-1,2,3-benzotriazin-4-one-3-oxy tris-(pyrrolidino)-phosphonium hexafluorophosphate (PDOP), 2-(benzotriazol-1-yloxy)-1,3-dimethyl-2-pyrrolidin-1-yl-1,3,2-diazaphospholidinium hexafluorophosphonate (BOMP), 5-(1H-7-azabenzotriazol-1-yloxy)-3,4-dihydro-1-methyl 2H-pyrrolium hexachloroantimonate (AOMP), (1H-7-azabenzotriazol-1-yloxy)tris(dimethylamino) phosphonium hexafluoroposphate (AOP), 5-(1H-Benzotriazol-1-yl)-3,4-dihydro-1-methyl 2H-pyrrolium hexachloroantimonate: N-oxide (BDMP), 2-bromo-3-ethyl-4-methyl thiazolium tetrafluoroborate (BEMT), 2-bromo-1-ethyl pyridinium tetrafluoroborate (BEP), 2-bromo-1-ethyl pyridinium hexachloroantimonate (BEPH), N-(1H-benzotriazol-1-ylmethylene)-N-methylmethanaminium hexachloroantimonate N-oxide (BOMI), N,N′-bis(2-oxo-3-oxazolidinyl)phosphinic chloride (BOP-Cl), 1-(1H-benzotriazol-1-yloxy)phenylmethylene pyrrolidinium hexachloroantimonate (BPMP), 1,1,3,3-bis(tetramethylene)fluorouronium hexafluorophosphate (BTFFH), chloro(4-morpholino)methylene morpholinium hexafluorophosphate (CMMM), 2-chloro-1,3-dimethyl-1H-benzimidazolium hexafluorophosphate (CMBI), 2-fluoro-1-ethyl pyridinium tetrafluoroborate (FEP), 2-fluoro-1-ethyl pyridinium hexachloroantimonate (FEPH), 1-(1-pyrrolidinyl-1H-1,2,3-triazolo[4,5-b]pyridin-1-ylmethylene)pyrrolidinium hexafluorophosphate N-oxide (HAPyU), O-(1H-benzotriazol-1-yl)-N,N,N′,N;-bis(pentamethylene)uronium hexafluorophosphate (HBPipU), O-(1H-benzotriazol-1-yl)-N,N,N0,N0-bis(tetramethylene)urinium hexafluorophosphate (HBPyU), (1H-7-azabenzotriazol-1-yloxy)tris(pyrrolidino)phosphonium hexafluorophosphate (PyAOP), bromotripyrrolidinophosphonium hexafluorophosphate (PyBrOp), chlorotripyrrolidinophosphonium hexafluorophosphate (PyClOP), 1,1,3,3-bis(tetramethylene) chlorouronium hexafluorophosphate (PyClU), tetramethylfluoromamidinium hexafluorophosphate (TFFH), triphosgene, triazine-based reagents [cyanuric chloride, cyanuric fluoride, 4-(4,6-dimethoxy-1,3,5-triazin-2-yl)-4-methylmorpholinium chloride (DMT-MM), 2-chloro-4,6-dimethoxy-1,3,5-triazine (CDMT)], bis(2-chlorophenyl) phosphorochloridate, diphenyl phosphorochloridate, diphenyl phosphoroazide (DPPA) and any combination thereof.

A currently preferred coupling reagent is 2-(1H-7-azabenztriazol-1-yl)-1,1,3,3-tetramethyluronium hexafluoro phosphate (HATU). Another currently preferred coupling reagent is 3,4-dihydro-1,2,3-benzotriazin-4-one-3-oxy tetramethyluronium hexafluorophosphate (HDTU). Another currently preferred coupling reagent is N,N′-bis(2-oxo-3-oxazolidinyl) phosphinic chloride (BOP-Cl) Another currently preferred coupling reagent is an organophosphoro halogenate or a pseudohalogenate such as diphenyl phosphorochloridate and diphenylphosphoroazide (DPPA). Another currently preferred coupling reagent is a halogeno tris(organo)phosphonium hexafluoro phosphate such as bromo tris(dimethylamino)phosphonium hexafluoro phosphate (BrOP), chlorotris(dimethylamino)phosphonium hexafluoro phosphate (ClOP), bromotripyrrolidinophosphonium hexafluorophosphate (PyBrOp) and chlorotripyrrolidinophosphonium hexafluorophosphate (PyClOP).

### Oligonucleotide Synthesis

The synthesis of the oligonucleotide is conducted by any known oligonucleotide synthetic approach, including a phosphate approach, an H-phosphonate approach, or a phosphite approach. A currently preferred method is the phosphonate method.

### Solid Support

The concept of solid phase synthesis was originally developed simultaneously by Merrifield and Letsinger for peptide chemistry and subsequently adapted to oligonucleotide synthesis by Letsinger. The solid support commonly used in oligonucleotide synthesis is controlled pore glass (CPG), 110 available from Proligo-Degussa.

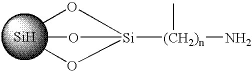

Solid Support for Oligonucleotide Synthesis

Polystyrene-copolymer supports have also been developed and are available commercially (for example, Primer Support from Pharmacia or polystyrene base solid supports from Glenn Research).

It was shown by the applicants that the resins developed for synthesis of peptides are also suitable for oligonucleotide synthesis (for example, PAM-resin or resins, containing fluoride anion cleavable linkers, described below). Using these resins, which having higher loading capacity than standard CPG support, it is possible to produce more oligonucleotides (g/per support unit) than using regular support.

The key step in oligonucleotide synthesis is the sequential stepwise formation of internucleotide phosphate bonds. The most common protecting groups for the nucleosides bases are benzoyl for adenine¹¹¹ and cytosine²⁵ and isobutyryl for guanine;²⁵ thymine usually does not require a protecting group. These groups are stable to all reagents used in oligonucleotide assembly steps.

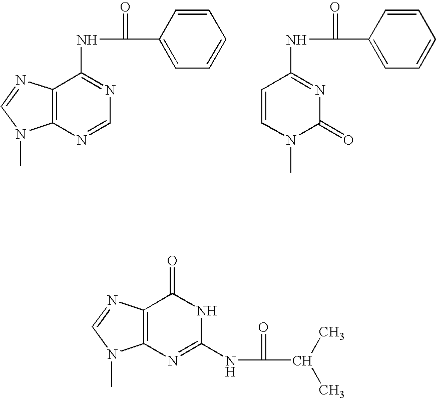

Exocyclic Amino Protecting Groups for Nucleoside Bases

These protecting groups are removed by treatment of ammonium hydroxide or mixture of ammonium hydroxide and methyl amine.

Although it has been reported that the aqueous ammonia treatment does not cause racemization or peptide bond cleavage, harsh ammonia conditions may lead to different side reactions such as a cleavage of linkers (for example, serine or tyrosine based) between peptide and oligonucleotide parts; base-catalyzed aspartimide formation in the synthesis of aspartic acid containing peptides, and many others.

To avoid undesirable side effects, the applicants have used the 9-fluorenylmethylcarbonyl (Fmoc) group for protection of the bases A, C and G during the synthesis of oligonucleotide-peptide conjugates. The advantage of Fmoc over the customary acyl blocking groups for A, C and G is that its removal in the final stage of the synthesis can be accomplished under conditions that leave the formed conjugate intact.

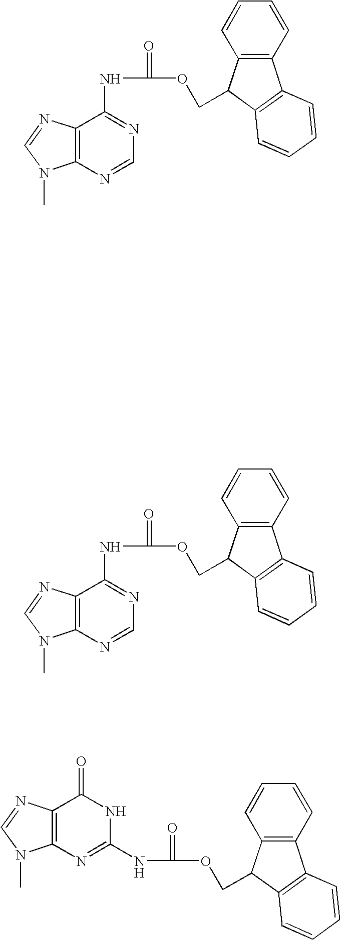

Fmoc-Protection for Nucleoside Bases

Because of the mild conditions of Fmoc removal, not only peptide-oligonucleotide conjugates, but different sensitive to base oligonucleotides with phosphate or thiophosphate chains can also be synthesized.

The 5′-hydroxyl group is protected by acid-labile ethers^{112,113} such as 4,4′-dimethoxytrityl (DMTr¹¹⁴ or 4-methoxytrityl (MMTr). These protecting groups are removed after each cycle by 3% dichloroacetic acid solution in dichloromethane.¹¹⁵

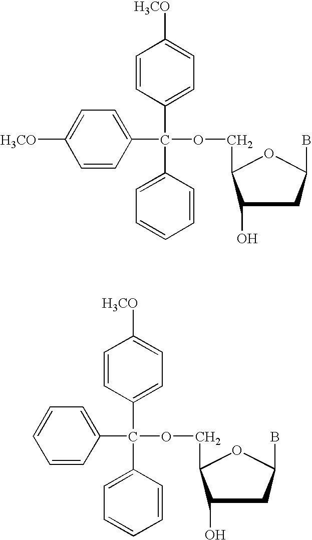

Protection of 5′-hydroxyl Group

Phosphitylating agents for nucleosides are summarized below:

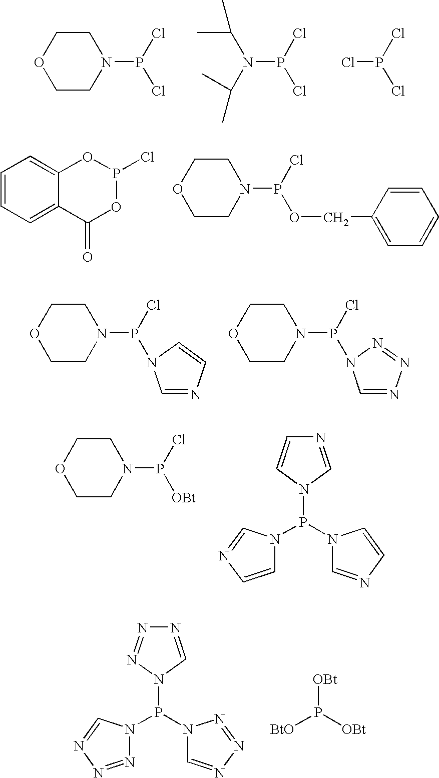

Phosphitylating Agents

### Oligonucleotide Synthesis by Phosphate Approach

This method was introduced in 1956 by H. G. Khorana¹¹⁶ and is outlined in Scheme 8. First, the DMT on the 5′-hydroxy position of the deoxyribonucleoside attached to the solid support is removed by 3% DCA. Next, the attached ODN reacts with an excess of protected 5′-dimthoxytrityl dioxyribonucleoside phosphate solution in the presence of a coupling reagent, such as N′N′-dicyclohexylcarbodiimide¹¹⁷ (DCC), mesitylenesulphonyl chloride,¹¹⁸ 2,4,6-triisopropylbenzenesulphonyl chloride¹¹⁹. At the end of the synthesis, the protecting groups on the ODN are cleaved by aqueous ammonia solution together with the ODN cleavage from the support.

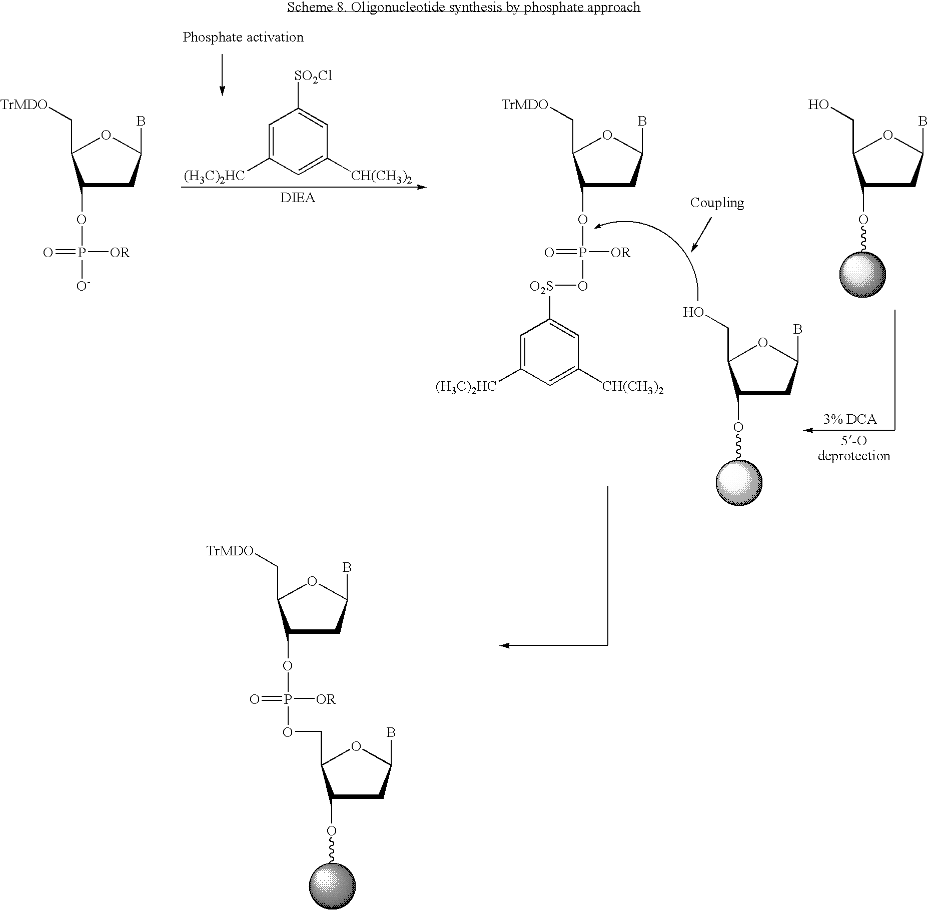

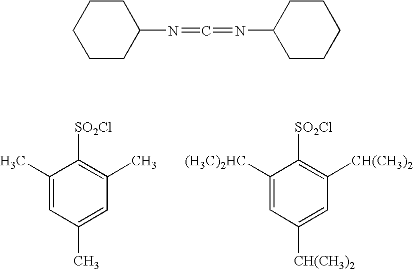

### Coupling Reagents for Phosphate Approach

The most useful protecting groups on the phosphate residue and their cleaving reagents are: 2-cyanoethyl¹²⁰ by β-elimination; 2,2,2-trichloroethyl by reduction with tributyl phosphine; benzoyl by hydrolysis in basic conditions; benzyl by Pd/H₂ reduction; and methoxymethane by treatment with thiol.

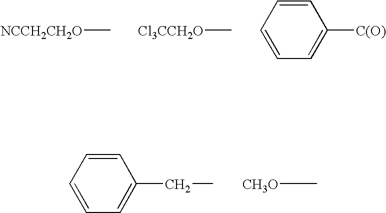

Phosphate Protecting Groups

### Oligonucleotide Synthesis by Phosphite Approach

Synthesis by phosphite method is outlined in scheme 9. The reactive species in this method are phosphoramidite.^{121,122} In the presence of a weak acid, like tetrazole (good leaving group formation), a phosphate bond is formed (after oxidation).

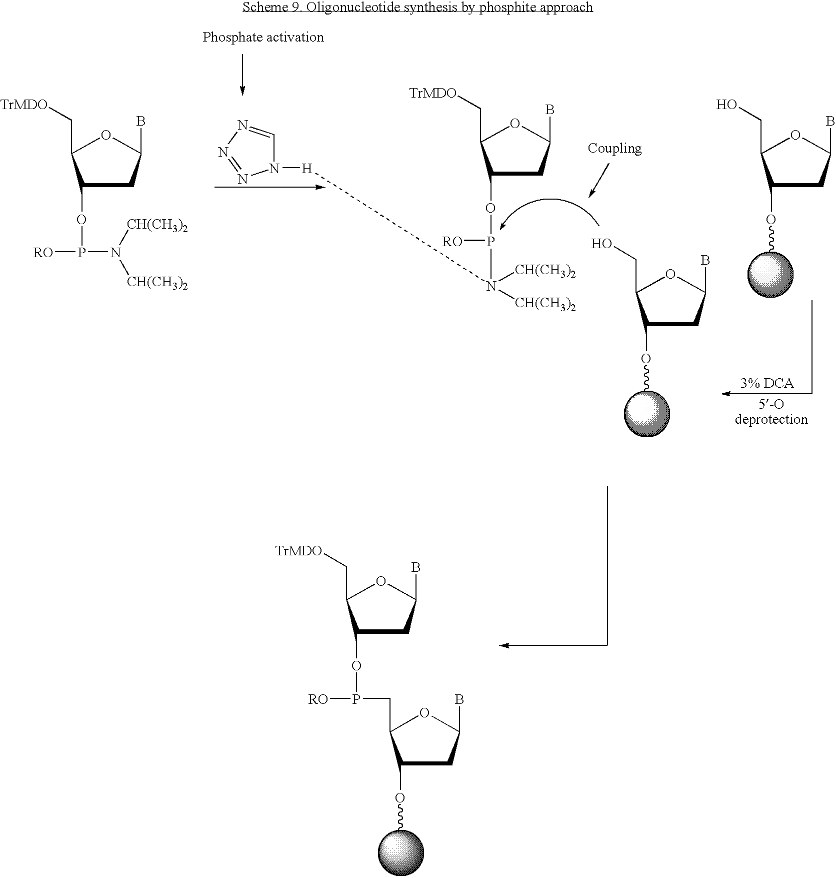

### Oligonucleotide Synthesis by H-phosphonate Approach

Oligonucleotide synthesis by H-phosphonate^{123,124} approach is outlined in Scheme 10. The monomer is activated by a hindered acyl chloride, the anhydride formed is used to react with a free oligonucleotide 5′-OH end, forming an H-phosphonate analogue of the internucleotidic linkage. Pivaloyl chloride and 1-adamantane carbonyl chloride were reported to be the suitable activators (yields are approximately 96-99%). Dipentafluorophenyl carbonate also provides high coupling ability, but is less reactive than pivaloyl chloride. At the end of the synthesis, all protecting groups are removed and the ODN is cleaved from the solid support by ammonia solution.

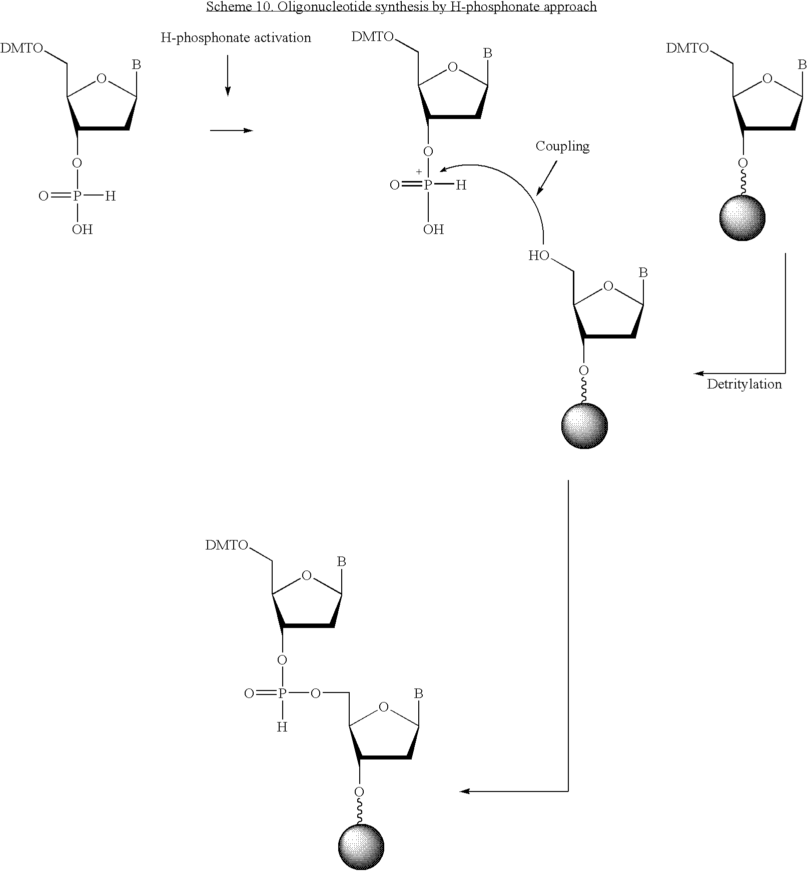

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXPERIMENTAL DETAILS SECTION

### Example 1

### Synthesis of Building Units

The major obstacles of sequential synthesis of peptide-ODN conjugate emanate from the inadequacy of peptide deprotection method with ODN stability. In the Fmoc and t-Boc approaches, side chain deprotections require strong acid that lead to depurination of the ODN: TFA for Fmoc and HF and TMSA for t-Boc. Therefore, in order to find a compatible method for the synthesis of the bipartite pathways, the commonly used synthetic approaches regarding α-amine and side chain protection of AA were modulated. A new strategy for a stepwise synthesis of peptide—oligonucleotide hybrid, which is based on the premise of appropriate protecting groups that will be cleaved under mild conditions, has been developed. The two types of protecting groups of amino acids involve either the α-amino site or the side chains.

α-amino Group Protection:

For protection of the α-amino group of AA, the NPS (p-nitrophenyl sulphenyl) residue, a well known protecting unit for amine and thiol function, was selected.¹²⁵ This unit can be removed by hydrogen chloride in methanol or by strong acids in aqueous methanol or acetone.¹²⁵ However, these conditions are “strong” enough to also remove most side-chain protecting groups or to destroy the ODN, if the synthesis of the conjugate starts from the oligonucleotide. Another method for removal of the Nps-group is to use triphenylphosphine (or tributylphosphine) and water in dioxane solution.¹²⁸ These conditions may also not be suitable for POC synthesis because of parallel removal of protecting group from cysteine, and due to the formation of a phosphine oxide byproduct which is difficult to remove.

The applicants of the present invention have found that the Nps-group is cleaved by solution of 1M thioacetamide in the presence of a catalytic amount of dichloroacetic acid. The applicants have further surprisingly and unexpectedly found that these conditions are so mild that all other protecting groups are unaffected.

Synthesis of the designated α-amino protected amino acid group is exemplified in Scheme 11A. The free amine of AA reacts with o-nitrophenyl sulphenyl chloride in basic condition (NaOH 2M). The desired protected amino acid is then precipitated by addition of 5% cold citric acid at pH=3-3.5.

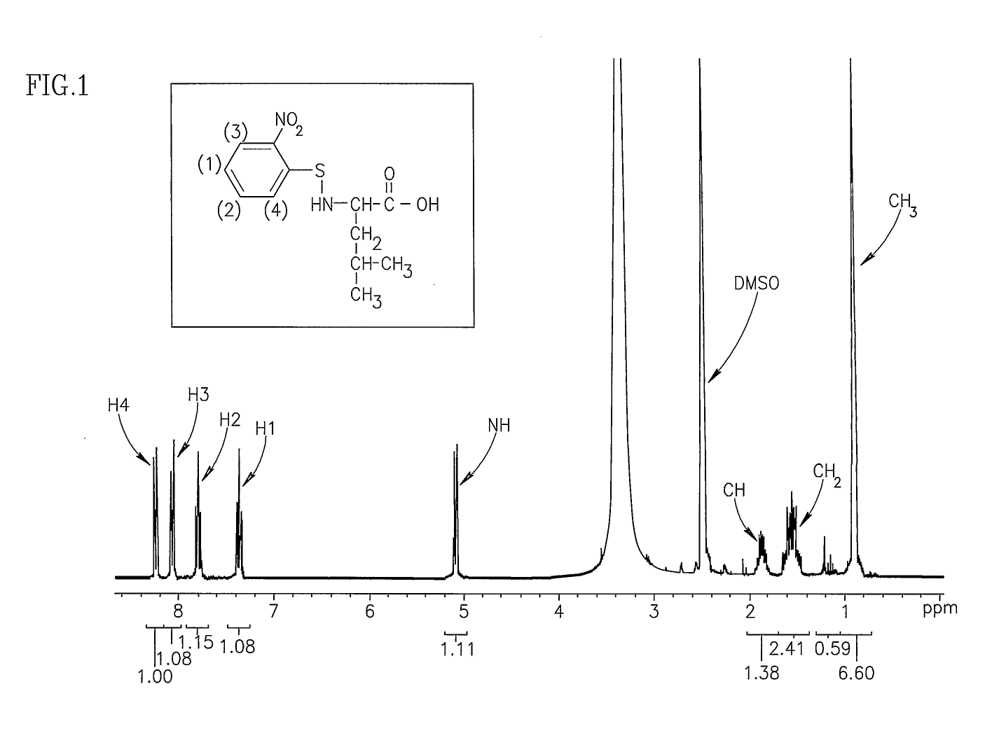

The following compounds were prepared in accordance with this method: Nps-Ala, Nps-Pro, Nps-Gly, Nps-Val, Nps-Gln, Nps-Leu, Nps-Ile in good yields (73-96%). NMR of these compounds shows the expected chemical shift of α-amine doublet at 5.1-5.2 ppm and four signals of the NPS group in the aromatic region of 7.3 to 8.4 ppm (see NMR spectra of NPS-Leu—FIG. 1).

### Side Chain Protecting Groups:

Suitable protecting groups for AA's side chains, that are compatible with the α-amine Nps-protecting group, were selected. Applicants selected a protecting group, which can be removed under mild conditions by fluoride anion, such as a silyl protecting group. The dimethyl-tert-butyl silyl (TBDMS) group (Scheme 11A) was selected as a suitable model to protect the oxygen of Thr. Deprotection takes place according Scheme 11B. This group can be successfully used to protect, e.g., the threonine and serine side chains.

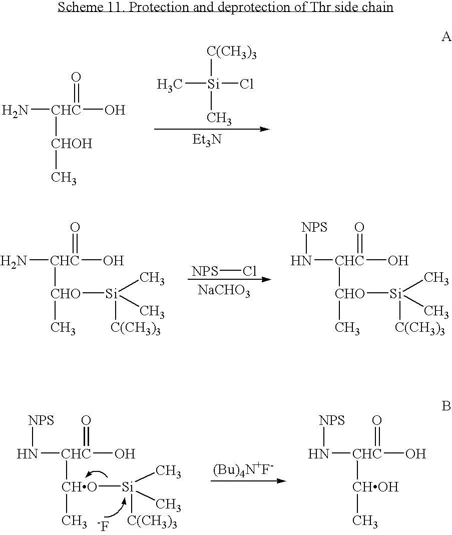

In addition to the known TBDMS protecting group, the applicants have surprisingly discovered a new silyl protecting group which contains a 4-trialkylsilyloxybenzylcarbonyl moiety, that can be removed under mild conditions and that can be used as a universal protecting group for AA side chains.

This novel side chain protecting group was introduced via a 4-nitrophenyl ester 4-triisopropylsilanoxybenzyl carbonate (BnSyl). The preparation is presented in Scheme 12A:

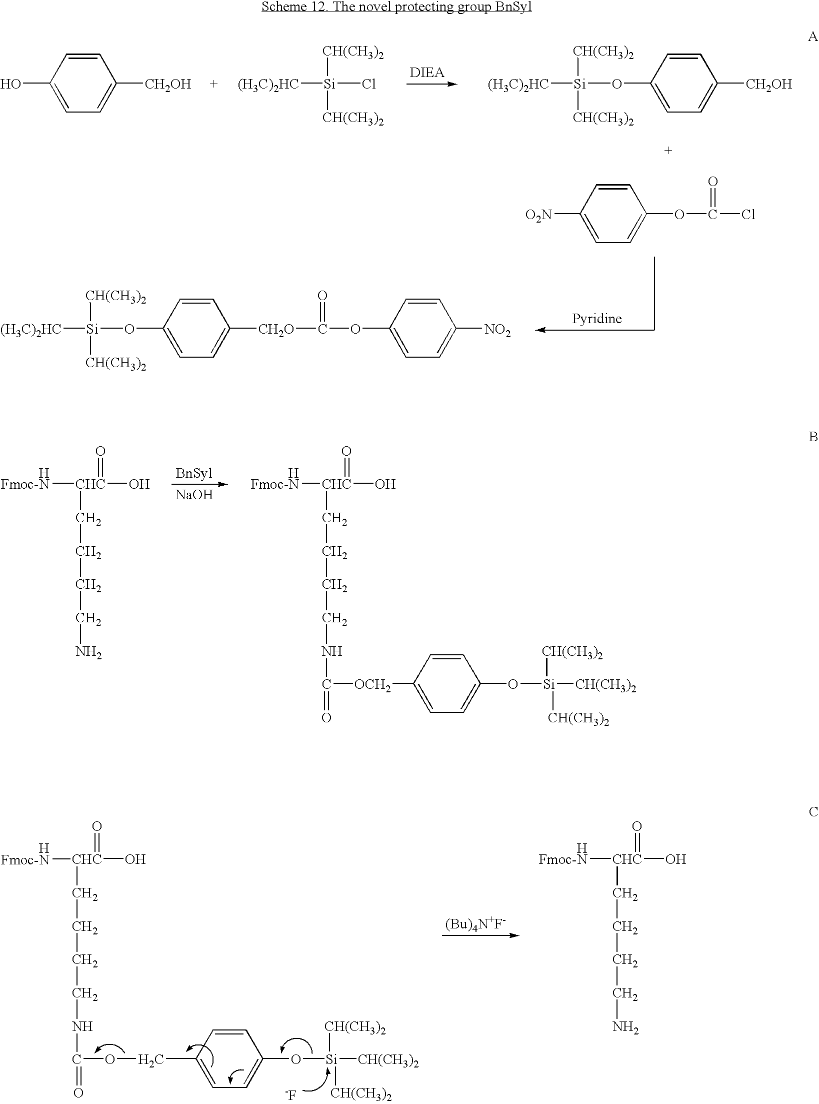

4-hydroxybenzyl alcohol was allowed to react with the triisopropylsylil chloride to give 4-hydroxysylilbenzyl alcohol. Due to the difference in the basicity between the phenol and benzyl alcohol, the silylation takes place exclusively on the phenolic group. The resulting product reacts with o-nitrophenyl chloroformate¹²⁷ to give the final material BnSyl. This novel protecting group was used to protect the ω-amine of Lys (Scheme 12B). Deprotection of ω-amine is achieved as shown above (Scheme 12C).

It is known that Fmoc and Fm groups can also be removed by fluoride anion.¹²⁹ Accordingly, in another experiment, the side chains of Lysine and Arginine were protected with Fmoc, in addition to protection of Asp and Glu as Fm-esters.

Preparation of protected Arg is carried out via a number of steps (Scheme 13A). Boc-Arg(Fmoc)₂-OH was prepared from Boc-Arg-OH.HCl by addition of 9-fluorenylmethoxycarbonyl chloride (Fmoc-Cl) in basic conditions (N,N′-diisopropylethyl amine). Then, the Boc group was removed by treatment with trifluoroacetic acid. Next, the Nps group was introduced on thea-amine as previously described. The crude product was purified by chromatography to give the required Nps-Arg(Fmoc)₂-OH. NMR and elementary analysis confirm the structure of product. The mechanism of Fmoc cleavage by fluoride ion via β-elimination (tetrabutyl ammonium fluoride for 1 hour) in presented in Scheme 13B.

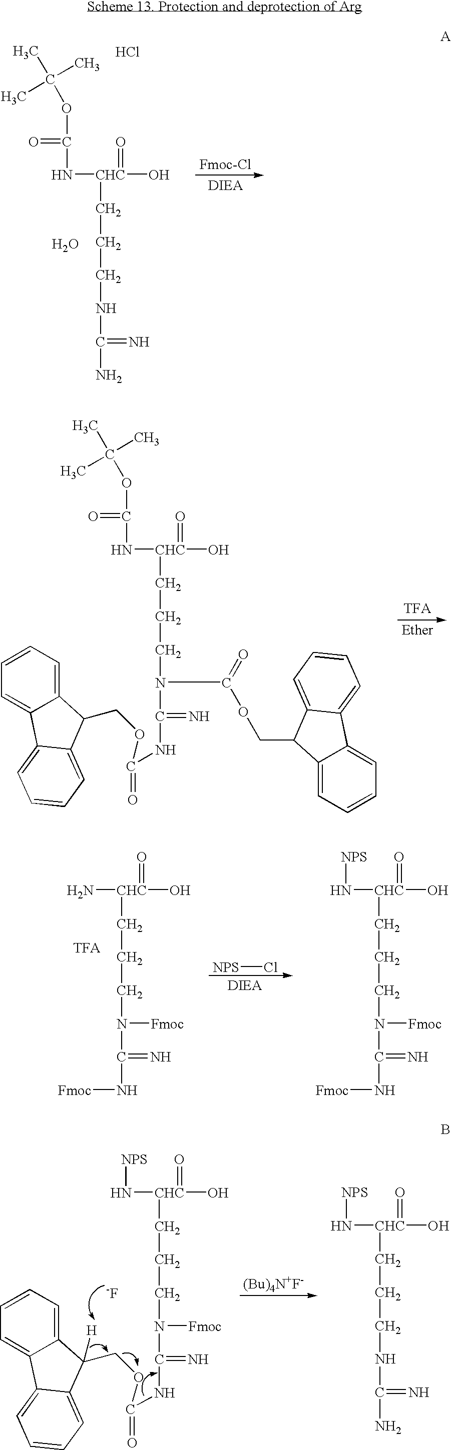

Asp derivative was prepared as is shown in Scheme 14A. The side chain was protected by 9-fluorenylmethanol (OFm) in the form of an ester¹²⁶ through the addition of 9-fluorenylmethanol to the amino acid under HBF₄ catalysis. The MW of the product was verified by MS-ES. The second step involved the protection of α-amine by the Nps group. The crude product was purified by chromatography. As already mentioned, the deprotection of side chain is effected by tetrabutylammonium fluoride, as shown in Scheme 14B. The same procedure was used for the preparation of a Glu derivative.

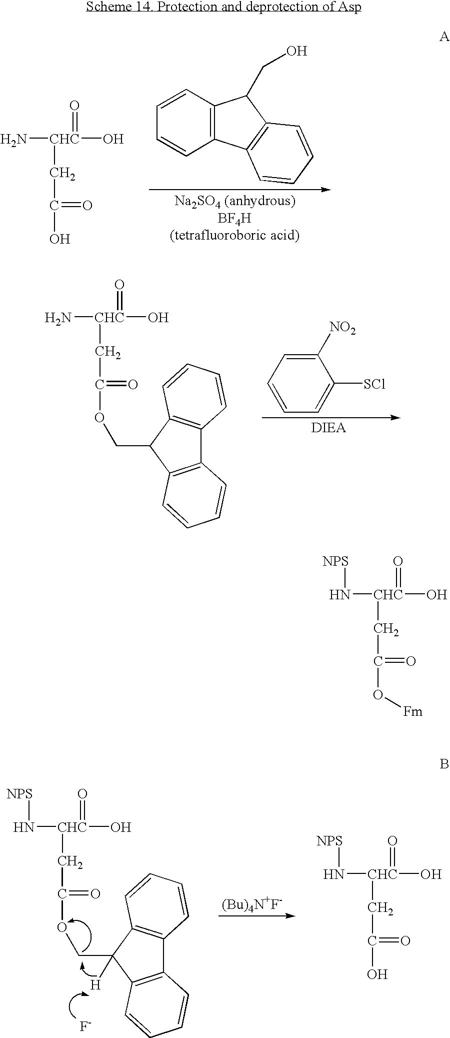

The Lysine side chain was also protected by an Fmoc group, as shown in Scheme 15A. In the first stage, TFA-Lys(Fmoc)-OH was prepared by treatment of Boc-Lys(Fmoc)-OH with trifluoroacetic acid to remove the t-Boc group from the α-amino group. Then, Nps was linked to the α-free amine by addition of o-nitrophenylsulphenyl chloride under basic conditions. The product, NPS-Lys(Fmoc)-OH was purified by chromatography. The side chain deprotection is performed as previously described (Scheme 15B).

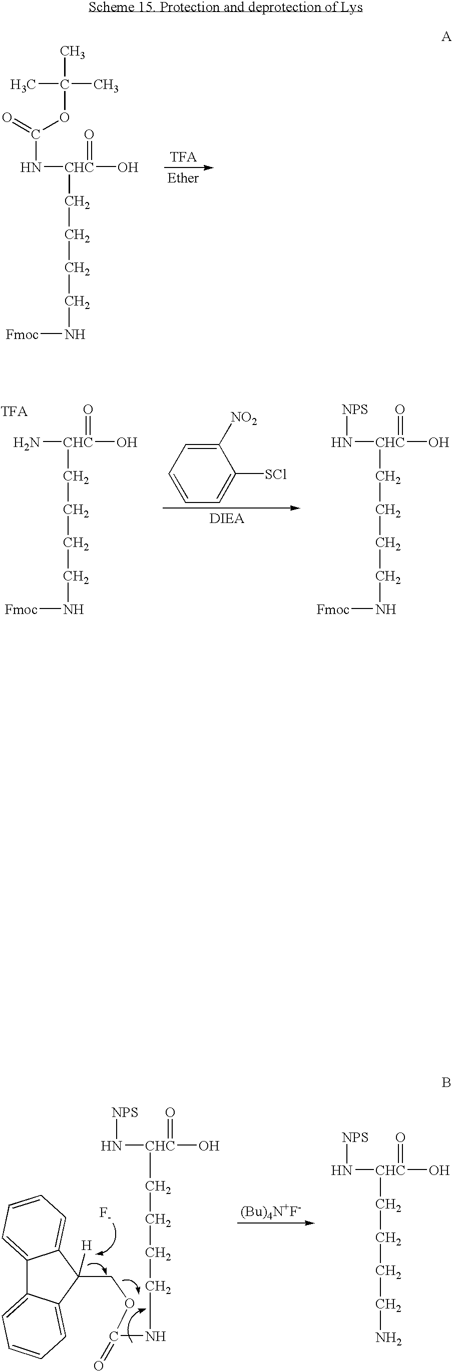

In summary, the applicants of the present invention have synthesized a range of protected amino acids with new combination of protected groups: Nps for α-amino function and TBDMS/BnSyl/Fmoc/Fm for side chains. This combination allows the synthesis of peptides under neutral mild conditions.

### Example 2

### Peptide Synthesis

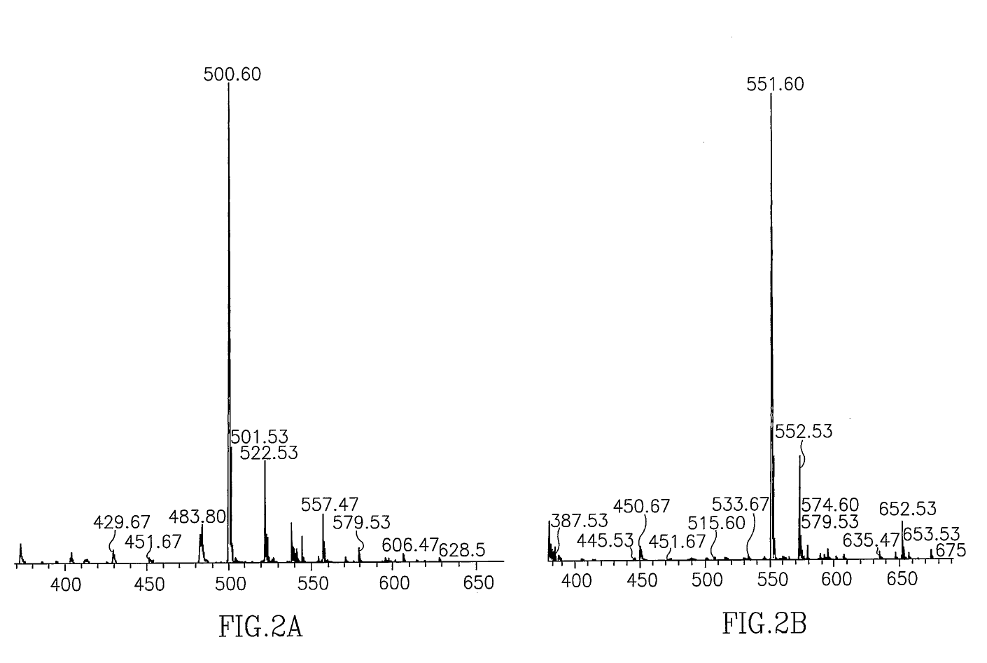

Using the building blocks described in Example 1, the applicants have synthesized two model peptides A) NH₂-Gln-Pro-Gly-Ala-Lys-OH (Mw=499.56 g/mol); and B) NH₂-Lys-Thr-Thr-Thr-Thr-OH (Mw=550.6 g/mol), which are both fragments of biologically active proteins (Scheme 16). After final deprotection and cleavage from resin, these peptides were purified by HPLC and their molecular weight confirmed by MS-ES (FIG. 2).

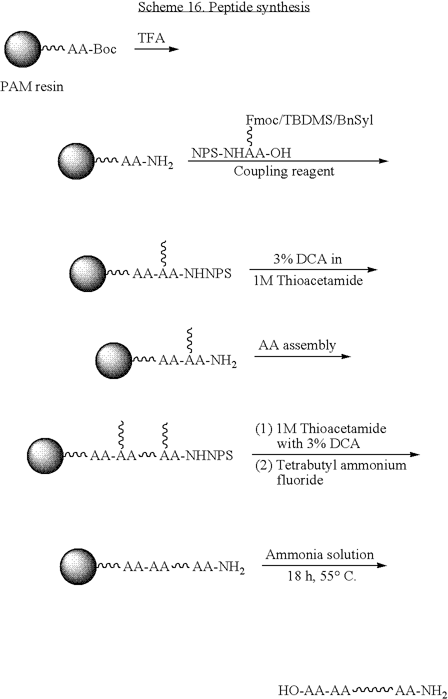

### Example 3

### Oligonucleotide Synthesis

Oligonucleotides were prepared using coupling reagents devised for peptide synthesis by a hydrogen phosphonate approach. The choice of the hydrogen phosphonate moiety as the phosphorylating reagent is based on its unique characteristics, namely a) relatively stability; b) it does not require protecting groups; and c) it is adequate for coupling with peptide coupling reagents as a monoacid.

The following hydrogen phosphonate nucleotides have been synthesized: protected adenosine (A^{bz}), cytosine (C^{bz}), thymine (T) and guanosine (G^{t-Bu}) phosphonates:

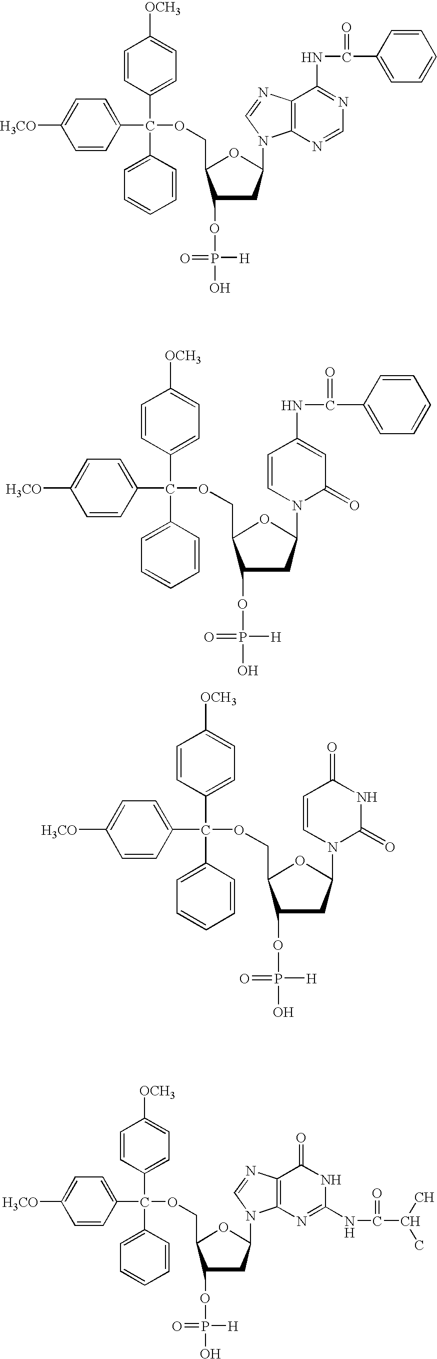

Building Units for Oligonucleotide Synthesis

All building units were prepared in the same manner by two step synthesis as shown in Scheme 17.

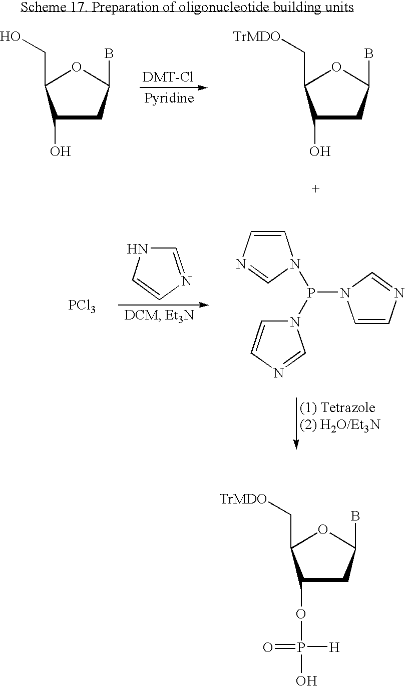

The 5′-hydroxyl group was protected by addition of dimethoxytrityl chloride to deoxyribonucleosides under basic conditions. The phosphonate at the 3′-OH position was introduced by treating the protected nucleoside with tri-(imidazole-1-yl) phosphine and an equivalent of 1H-tetrazole, followed by addition of water. The structure of the phosphonate was confirmed by ³¹P-NMR spectroscopy. The yields were 90-95%.

### Example 4

### Preparation of Peptide-Oligonucleotide Conjugate (POC)

POC were synthesized according to following scheme 18:

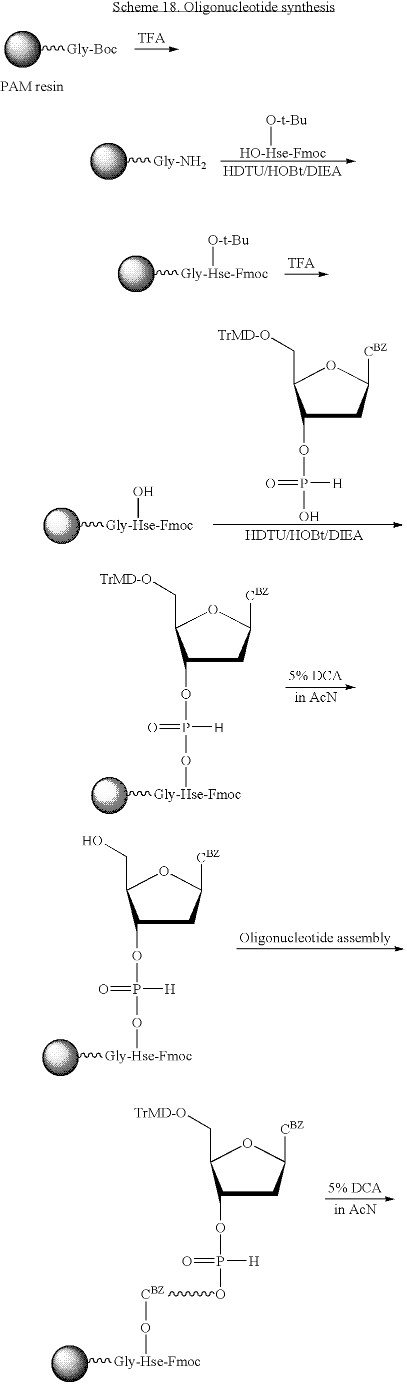

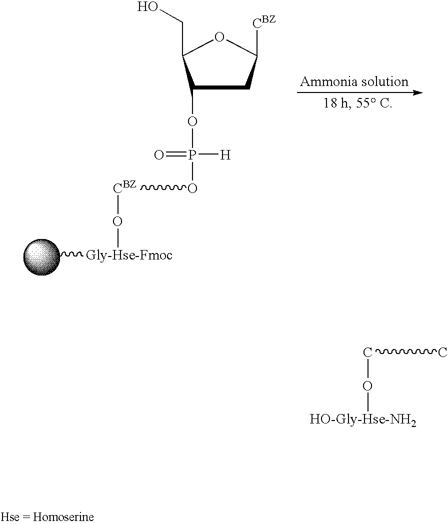

### Summary

In summary, the applicants of the present invention have developed a new methodology of peptide synthesis under mild neutral condition on solid support. A) For this purpose new peptide building blocks were prepared. B) New mild conditions for removal of Nps group (thioacetamide/dichloroacetic acid) were discovered. C) protecting units for AA's side-chains were identified and selected, which are orthogonal to (compatible with) the Nps-group ((R₄)Si, BnSyl, Fmoc and Fm). In particular, it was shown that Fmoc and Fm side-chain protecting units are stable in acidic media and can be easily removed by fluoride anion under neutral conditions. D) Using the new combination of Nps and Fmoc/Fm protecting groups permitted the synthesis of desired peptides in good yield and satisfactory purity. E) Different coupling reagents (HBTU, BOP, DCC, HATU, HDTU, PDOP) were tested in peptide synthesis.

It was also found that the combination of H-phosphonate approach using coupling reagents (e.g., HDTU, HATU, BOP-Cl, PyBrOP, PyClOP, ClOP, BrOP, diphenylphosphorochloridate) serves an effective method for ODN synthesis, which is compatible with the synthesis of peptides.

A new method of peptide-oligonucleotide conjugate synthesis under mild conditions on solid support was thus developed. This method can be performed manually or by synthesizer and can be found an application in the synthesis of various peptide-oligonucleotide conjugates, especially base-or acid sensitive, constructed from alternate peptide and oligonucleotide blocks, branched and cyclic.

### Example 5

### Experimental Procedures

### A. Abbreviations

Acetonitrile: ACN; t-Butyldimethylsilyl chloride: TBDMSCl; Dichlioroacetic acid: DCA; Dimethoxytril chloride: DMT-Cl; N,N′-Diisopropylethylanime: DIEA; Triethylamine: Et₃N; Dichloromethane: DCM; Mass spectrometry—electro spray: MS-ES; Nuclear magnetic resonance: NMR; Singlet: s; Doublet: d; Double Doublet: dd; Triplet: t; Multiplet: m; Magnesium sulfate: MgSO₄; o-Nitrophenylsulphenyl chloride: NPS-Cl; Room temperature: rt.; Tetrabutylammonium fluoride: BuN⁺F⁻; Trifluoroacetic acid: TFA; 9-fluorenylmethoxycarbonyl chloride: Fmoc-Cl; 9-fluorenylmethanol: Fm-OH; Trimethylchlorosilane: TMS-Cl; N,N′-Dimethyl formamide: DMF; Sodium sulphate: Na₂SO₄; Sodium hydroxide:NaOH; N-methylpyrrolidone: NMP; Dimethyl sulfoxide: DMSO

### B. General

Proton nuclear magnetic resonance (¹H NMR) spectra were recorded on VXR-300S Varian spectrometer, using DMSO protons as the internal standard. Phosphorus NMR (³¹P NMR) spectra were recorded on a 121.4 MHz spectrometer, using phosphoric acid as the external standard.

High-performance liquid chromatography (HPLC): Analytical and preparative (C₁₈) column chromatography was used. ACN/0.1% TFA and H₂O/0.1% TFA were used as the eluents.

### C. Synthesis

### Preparation of Nps-AA

15 mmol amino acid was dissolved in a mixture of 10 ml of 2 NNaOH and 25 ml of dioxane. During a period of 30 min, 17.1 mmol of Nps-Cl and 2 NNaOH (10 ml) were added in 10 equal portions, with vigorous stirring. After 3 hours the solution was diluted with 50 ml of water, filtered, and acidified with cold 5% citric acid. The syrupy precipitate usually crystallized upon scratching and cooling. The product was filtered off, washed with water, dried, dissolved in ethyl acetate, and precipitated again by addition of petroleum ether.

### Nps-Ala (41)

Mp. 74-76° C.

Yield 2.9976 g (82.5%).

Anal. Calcd. for C₉H₁₀N₂O₄S: C, 44.62; H, 4.16; N, 11.56; S, 13.24. Found: C, 43.46; H, 3.38; N, 9.73; S, 14.78.

¹H NMR (DMSO-d₆, δ): 8.254-8.225 (d, 1H, Ph ortho to NO₂); 7.998-7.978 (d, 1H, Ph ortho to S); 7.805-7.774 (t, 1H, Ph meta to NO₂); 7.380-7.331 (t, 1H, Ph meta to S); 5.148-5.124 (d, 1H, N^{□}H); 3.487-3.440 (m, 1H, NH—CH—COOH); 1.342-1.319 (d, 3H, CH₃—CH).

### Nps-Pro (42)

Mp. 96-98° C.

Yield 3.5263 g (87.6%).

Anal. Calcd. for C₁₁H₁₂N₂O₄S: C, 49.24; H, 4.51; N, 10.44; S, 11.95. Found: C, 48.48; H, 4.14; N, 9.66; S, 12.58.

¹H NMR (DMSO-d₆, δ): 8.272-8.246 (d, 1H, Ph ortho to NO₂); 7.848-7.751 (m, 2H, 1H, Ph ortho to S and Ph meta to NO₂); 7.406-7.350 (t, 1H, Ph meta to S); 3.897-3.857 (d, 1H, CH₂—CH—COOH); 1.964 (br, 4H, CH—CH₂—CH₂—CH₂).

### Nps-Gly (43)

Mp. 120-122° C.

Yield 3.184 g (93.01%).

Anal. Calcd. for C₈H₈N₂O₄S: C, 42.1; H, 3.53; N, 12.27; S, 14.05. Found: C, 42.31; H, 3.45; N, 11.92; S, 14.5.

¹H NMR (DMSO-d₆, δ): 8.253-8.225 (d, 1H, Ph ortho to NO₂); 7.991-7.964 (d, 1H, Ph ortho to S); 7.815-7.760 (t, 1H, Ph meta to NO₂); 7.383-7.327 (t, 1H, Ph meta to S); 5.098-5.079 (d, 1H, N^{□}H); 1.207 (s, 2H, NH—CH₂—COOH).

### Nps-Val (44)

Mp. 75-77° C.

Yield 3.5242 g (86.9%).

Anal. Calcd. for C₁₁H₁₄N₂O₄S: C, 48.88; H, 5.22; N, 10.36; S, 11.86. Found: C, 47.98; H, 4.75; N, 9.85; S, 12.17.

¹H NMR (DMSO-d₆, δ): 8.253-8.225 (d, 1H, Ph ortho to NO₂); 8.082-8.050 (d, 1H, Ph ortho to S); 7.815-7.760 (t, 1H, Ph meta to NO₂); 7.383-7.327 (t, 1H, Ph meta to S); 5.018-4.988 (d, 1H, N^{□}H); 3.143-3.094 (q, 1H, NH—CH—COOH); 2.088-2.023 (m, 1H, CH—CH—CH₃); 1.009-0.973 (q, 6H, CH—CH₃).

### Nps-Gln (45)

Mp. 153-157° C.

Yield 4.3009 g (96.5%).

Anal. Calcd. for C₁₁H₁₃N₃O₅S: C, 44.14; H, 4.38; N, 14.04; S, 10.71. Found: C, 43.83; H, 4.23; N, 13.22; S, 10.74.

¹H NMR (DMSO-d₆, δ): 8.255-8.223 (d, 1H, Ph ortho to NO₂); 8.080-8.048 (d, 1H, Ph ortho to S); 7.807-7.751 (t, 1H, Ph meta to NO₂); 7.391-7.336 (t, 1H, Ph meta to S); 6.782 (s, 2H, CO—NH₂); 5.119-5.092 (d, 1H, N^{a}H); 2.304-2.222 (q, 2H, CH₂—CH₂—CO); 1.979-1.811 (m, 2H, CH—CH₂—CH₂).

### Nps-Leu (46)

Mp. 93-95° C.

Yield 1.0439 g (73.5%).

Anal. Calcd. for C₁₂H₁₆N₂O₄S: C, 50.69; H, 5.67; N, 9.85; S, 11.28. Found: C, 50.4; H, 5.57; N, 9.77; S, 10.84.

¹H NMR (DMSO-d₆, δ): 8.253-8.221 (d, 1H, Ph ortho to NO₂); 80.79-8.048 (d, 1H, Ph ortho to S); 7.813-7.758 (t, 1H, Ph meta to NO₂); 7.383-7.328 (t, 1H, Ph meta to S); 5.092-5.064 (d, 1H, N^{a}H); 1.891-1.821 (m, 1H, CH₂—CH—CH₃); 1.591-1.501 (m, 2H, CH—CH₂—CH); 0.899-0.873 (d, 6H, CH—CH₃).

### Nps-Ile (47)

Mp. 59-61° C.

Yield 0.7588 g (53.4%).

Anal. Calcd. for C₁₂H₁₆N₂O₄S: C, 50.69; H, 5.67; N, 9.85; S, 11.28. Found: C, 50.8; H, 5.48; N, 9.54; S, 10.85.

¹H NMR (DMSO-d₆, δ):8.246-8.219 (d, 1H, Ph ortho to NO₂); 8.061-8.033 (d, 1H, Ph ortho to S); 7.828-7.753 (t, 1H, Ph meta to NO₂); 7.377-7.325 (t, 1H, Ph meta to S); 4.978-4.95 (d, 1H, N^{a}H); 1.562-1.476 (m, 1H, NH—CH—COOH); 1.322-1.224 (m, 2H, CH—CH₉—CH₃); 0.958-0.936 (d, 3H, CH—CH₃); 0.880-0.932 (t, 3H, CH₂—CH₃).

### Preparation of NPS-Thr(O-DMTBS)-OH (48)

Preparation of Thr(O-DMTBS)-OH (A)

To a solution of 1.19 g (10 mmol) of L-threonine in DCM and ACN (1:1) 35 mmol of Et₃N and 1.81 g (12 mmol) of TBDMS-Cl were added. The mixture was refluxed overnight. All solvents were evaporated in vacuo and the reaction residue was re-dissolved in DCM and ACN. To this reaction mixture 15 mmol of Et₃N and 0.902 g (6 mmol) of TBDMS-Cl were added. The mixture was refluxed overnight then evaporated in vacuo to get a white solid. The crude product was dissolved in DCM, washed several times with water, dried (Na₂SO₄), and evaporated to yield a white solid A.

Mp. 155° C.

MS-ES m/z [M+H]⁺: 234.27. Calcd. 233.38.

Preparation of Nps-Thr(O-DMTBS)-OH (B).

To a solution of 1.166 g (5 mmol) of A in ACN and 10 mmol of bicarbonate solution was added Nps-Cl in small portions over a period of 30 min. After 3 hours the solution was diluted with 50 ml of water, filtered, and acidified with cold 5% citric acid. The precipitate formed was filtered, washed with water, dried, dissolved in ethyl acetate, and precipitated again by addition of petroleum ether to yield 1.17 g (61%) of B.

Mp. 108-110° C.

Anal. Calcd. for C₁₆H₂₆N₂O₅SSi: C, 49.72; H, 6.78; N, 7.25; S, 8.30. Found: C, 49.12; H, 6.73; N, 7.08; S, 7.75.

### Preparation of NPS-Arg(Fmoc)₂-OH (49)

(1) Preparation of Boc-Arg(Fmoc)₂-OH(C)

5 g (15 mmol) of Boc-Arg-OH.HCl was co-evaporated three times with dry ACN. Then 125 ml of DCM was added, followed by 10.5 ml of DIEA and 9 ml of TMS-Cl. The reaction mixture was refluxed under nitrogen for 90 min, and then cooled. 8 ml of DIEA and 12 g of solid Fmoc-Cl were added. After stirring for 30 min in cold bath the temperature was elevated to rt, and the reaction mixture was stirred for an additional for 4 hours. The solution was then washed several times with water, dried over sodium sulfate, filtered and evaporated in vacuo. The crude product (7.3 g) was purified on silica gel column (dichloromethane:methanol; 95:5) to yield 7.3 g (67%) of C.

(II) Preparation of TFA•Arg(Fmoc)₂-OH (D)

Compound C was dissolved in 20 ml concentrated TFA, and the reaction mixture was stirred for 30 min. The product D was precipitated by addition of ether, filtered, washed with ether and dried over phosphorous pentoxide in vacuo.

MS-ES m/z [M+H]⁺: 619.40. Calcd. 618.68.

(III) Preparation of Nps-Arg(Fmoc)₂-OH (E)

To a solution of D (1.46 g, 2 mmol) in 10 ml DMF 1.3 ml (7.5 mmol) of DIEA and 0.34 g (1.8 mmol) of NPS-Cl were added. The mixture was stirred 90 min and then diluted with ethyl acetate. The reaction mixture was acidified with 5% citric acid, washed with brine, water, dried over sodium sulfate, and evaporated to a small volume. The crude product (1.38 g) was precipitated by addition of petroleum ether. It was then purified on preparative HPLC to yield 0.86 g (47.3%) of E.

Mp. 85-87° C.

Anal. Calcd. for C₄₂H₃₇N₅O₈S: C, 65.36; H, 4.83; N, 9.07; S, 4.15. Found: C, 61.41; H, 4.64; N, 8.36; S, 4.21.

¹H NMR (DMSO-d₆, δ): 8.225-8.193 (d, 1H, Ph ortho to NO₂); 8.013-7.986 (d, 1H, Ph ortho to S); 7.868-7.555 (m, 8H, H4+H5+H1+H8 of Fmoc); 7.399-7.216 (m, 10H, Ph meta to NO₂ and Ph meta to S and H2+H3+H6+H7 of Fmoc); 5.036-5.010 (d, 1H, N^{a}H); 4.716-4.700 (d, 2H, CH₂ of Fmoc); 4.410-4.368 (t, 1H, H9 of Fmoc); 4.170 (br, 1H, NH—CH—COOH); 1.422 (br, 4H, CH—CH₂—CH₂—CH₂).

### Preparation of Nps-Lys(Fmoc)-OH (50)

Preparation of Lys(Fmoc)-OH (F)

The solution of Boc-Lys(Fmoc)-OH in 15 ml TFA was stirred for 4 hours. The product was precipitated by addition of cold ether then dried over P₂O₅ in vacuo.

MS-ES m/z [M+H]⁺: 369.65. Calcd. 368.43.

### Preparation of Nps-Lys(Fmoc)-OH (G)

2.46 g (5 mmol) of F was dissolved in a solution of 5 ml of DIEA and 25 ml of dioxane. During a period of 30 min 1.14 g (6 mmol) of Nps-Cl and 2.5 ml of DIEA were added dropwise with vigorous stirring. After 3 hours the solution was evaporated. The crude product was purified by preparative HPLC to yield 1.7 g (65%) of G.

Mp. 135-137° C.

Anal. Calcd. for C₂₇H₂₇N₃O₆S: C, 62.17; H, 5.22; N, 8.06; S, 6.15. Found: C, 59.33; H, 5.14; N, 7.37; S, 6.34.

¹H NMR (DMSO-d₆, δ): 8.245-8.217 (d, 1H, Ph ortho to NO₂); 8.055-8.026 (d, 1H, Ph ortho to S); 7.869-7.844 (d, 2H, H4 and H5 of Fmoc); 7.779-7.685 (t, 1H, Ph meta to NO₂); 7.669-7.644 (d, 2H, H1 and H8 of Fmoc); 7.405-7.270 (m, 5H, Ph meta to S and H2+H3+H6+H7 of Fmoc); 5.088-5.062 (d, 1H, N^{□}H); 4.246-4.178 (m, 3H, CH₂ and H9 of Fmoc), 3.884 (br, 1H, NH—CH—COOH); 2.958-2.940 (d, 2H, CH₂—CH₃—NH); 2.0282 (s, 2H, CH₂—CH₂—CH); 1.732-1.727 (m, 4H, CH₂—CH₂—CH₂—CH₂).

### Preparation of Nps-Glu(Fm)-OH (52)

(I) Preparation of Glu(Fm)-OH (I)

To a suspension of 2.94 g (20 mmol) Glu-OH, 20 g (170 mmol) of 9-fluorenylmethanol, and 5 g of anhydrous Na₂SO₄ in 30 ml dry THF was added 85 mmol of tetrafluoroboric acid diethyletherate. The reaction mixture was stirred at rt for 14 h. The solution was then diluted with THF (60 ml) and filtered through celite. To the solution were added 9 ml DIEA, followed by 140 ml ethyl acetate. After overnight in 0° C., the crystals were filtered and washed with acetone and water to yield 4.9 g (75%) of I.

MS-ES m/z [M+H]⁺: 326.53. Calcd. 325.36.

1H NMR (DMSO-d₆, δ): 7.899-7.834 (d, 2H, H4 and H5 of Fm); 7.659-7.637 (d, 2H, H1 and H₈ of Fm); 7.436-7.261 (m, 4H, H2+H3+H6+H7 of Fm); 4.377-4.355 (d, 2H, CH₂ of Fm); 4.275-4.229 (t, 1H, H9 of Fm); 2.004-1.838 (m, 4H, CH—CH₂—CH₂—CO).

(II) Preparation of Nps-Glu(Fm)-OH (J)

2 g (6.16 mmol) of I were suspended in 50 ml water and 40 ml acetone. 1.3 ml (7.6 mmol) DIEA was added followed by 1.4 g (7.4 mmol) Nps-Cl with vigorous stirring. 1 ml DIEA was added and the pH was adjusted to 8.5. The mixture was stirred at rt for 1 h, and then 50 ml ethyl acetate were added. The mixture was acidified with 5% citric acid. The organic layer was separated and washed with 5% citric acid, brine, water, dried (Na₂SO₄), and the solvent was evaporated under reduced pressure to a small volume. The product was precipitated by addition of petroleum ether to yield J, 2.57 g (87%).

Mp. 135-137° C.

¹H NMR (DMSO-d₆, δ): 8.246-8.219 (d, 1H, Ph ortho to NO₂); 8.061-8.033 (d, 1H, Ph ortho to S); 7.899-7.834 (d, 2H, H₄ and H₅ of Fm); 7.828-7.753 (t, 1H, Ph meta to NO₂); 7.659-7.637 (d, 2H, H1 and H8 of Fm); 7.436-7.261 (m, 5H, Ph meta to S and H2+H3+H6+H7 of Fm); 4.377-4.355 (d, 2H, CH₂ of Fm); 4.275-4.229 (t, 1H, H9 of Fm); 2.004-1.838 (m, 4H, CH—CH₂—CH₂—CO).

### Preparation of Nps-Asp(Fm)-OH (51)

(I) Preparation of Asp(Fm)-OH (K)

The procedure is as for 1 except that the reaction mixture was heated at 60° C. for 12 h.

Yield of K is 2.44 g (39%).

MS-ES m/z [M+H]⁺: 312.53. Calcd. 311.33.

¹H NMR (DMSO-d₆, δ): 7.906-7.881 (d, 2H, H4 and H5 of Fm); 7.685-7.661 (d, 2H, H1 and H8 of Fm); 7.444-7.305 (m, 4H, H2+H3+H6+H7 of Fm); 4.365-4.291 (m, 3H, H9 and CH₂ of Fm); 2.963-2.888 (m, 1H, NH—CH—COOH); 2.688-2.604 (m, 2H, CH—CH₂—CO).

(II) Preparation of NPS-Asp(Fm)-OH (L)

The procedure is as for J.

Yield 0.4 g (86%).

Mp. 112-114° C.

¹H NMR (DMSO-d₆, δ): 8.253-8.221 (d, 1H, Ph ortho to NO₂); 8.091-8.052 (d, 1H, Ph ortho to S); 7.906-7.881 (d, 2H, H2 and H9 of Fm); 7.685-7.661 (d, 2H, H6 and H5 of Fm); 7.812-7.753 (t, 1H, Ph meta to NO₂); 7.444-7.305 (m, 5H, Ph meta to S and H3+H4+H7+H8 of Fm); 4.365-4.291 (m, 3H, H9 and CH₂ of Fm); 2.963-2.888 (m, 1H, NH—CH—COOH); 2.688-2.604 (m, 2H, CH—CH₂—CO).

### Carbonic acid 4-nitrophenyl ester 4-triisopropylsilanoxybenzyl ester (BnSyl) (53)

(1) Preparation of (4-Triisopropylsilanyloxy-phenyl)-methanol (M)

To a solution of 24.8 g (200 mmol) 4-hydroxybenzyl alcohol in dichloromethane were added 75 mmol DIEA and 42.8 g (200 mmol) triisopropylsilyl chloride. The mixture was stirred overnight at rt. The reaction mixture was evaporated to yield a yellow oil mass (99.95 g). The product M was purified by column chromatography (dichloromethane:petroleum ether; 50:50). Yield 52.25 g (93.2%).

¹H NMR (DMSO-d₆, δ): 7.182-7.154 (d, 2H, Ph meta to CH₂); 6.799-6.772 (d, 2H, Ph ortho to CH₂); 5.045 (t, 1H, CH₂—OH); 4.402-4.384 (d, 2H, Ph-CH₂—OH); 1.235-1.162 (m, 3H, CH—Si); 1.049-1.025 (d, 18H, CH—CH₃).

(II) Preparation of Carbonic acid 4-nitrophenyl ester 4-triisopropylsilanoxybenzyl Ester (BnSyl) (N)

To a solution of 14.024 g (50 mmol) of M in dry THF/dichloromethane under nitrogen atmosphere were added, with stirring at 0° C., 22.65 g (1.5 eq) of 4-nitrophenylchloroformate and 6 ml of dry pyridine. The mixture was then stirred at rt for 72 hours, followed by addition of ethyl acetate. The organic layer was washed with 10% citric acid, brine, water, dried (Na₂SO₄), and evaporated to yield an yellow oil mass. The product N was purified by column chromatography (dichloromethane:petroleum ether; 70:30). Yield 15.9876 g (71.9%).

Anal. Calcd. for C₂₃H₃₁NO₆Si: C, 62.0; H, 7.01; N, 3.14. Found: C, 62.91; H, 7.42; N, 2.76. ¹H NMR (DMSO-d₆, δ): 8.308-8.277 (d, 2H, Ph ortho to NO₂); 7.555-7.525 (d, 2H, Ph meta to NO₂); 7.367-7.339 (d, 2H, Ph ortho to CH₂); 6.898-6.873 (d, 2H, Ph ortho to CH₂); 5.211 (s, 2H, Ph-CH₂—O); 1.263-1.191 (m, 3H, CH—Si); 1.057-1.033 (d, 18H, CH—CH₃).

### Preparation of Fmoc-Lyz(ZSyl)-OH (54)

To a solution of 2.46 g (5 mmol) Fmoc-Lys-OH in 30 ml dioxane, 2.6 ml DIEA and 1.14 g (6 mmol) of N were added. The reaction mixture was stirred overnight and then evaporated in vacuo. The crude product was purified by preparative HPLC to yield 2.88 g (64%).

Mp. 91-93° C.

¹HNMR (DMSO-d₆, δ): 7.880-7.855 (d, 2H, Fmoc); 7.712-7.686 (d, 2H, Fmoc); 7.414-7.193 (m, 6H, Ph meta to CH₂ and Fmoc); 6.819-6.796 (d, 2H, Ph ortho to CH₂); 4.882 (s, 2H, Ph-CH₂—O); 4.261-4.192 (m, 3H, H9 and CH₂ of Fmoc); 3.884 (br, 1H, NH—CH—COOH); 2.958-2.940 (d, 2H, CH₂—CH₂—NH); 2.0282 (s, 2H, CH—CH₂—CH₂); 1.732-1.727 (m, 4H, CH₂—CH₂—CH₂—CH₂); 1.230-1.158 (m, 3H, CH—Si); 1.029-1.005 (d, 18H, CH—CH₃).

### Peptide Chain Synthesis

Deprotection of first AA bonded to resin: (1) Fmoc-Amino Acid on TGA Resin (1 eq) was treated with a solution of piperidene 20% in NMP for 30 min and then was washed with NMP, DCM, and methanol; or (2) Boc-amino acid on PAM Resin (1 eq) was treated with trifluoroacetic acid for 30 min and then was washed with, Et₃N, NMP, DCM, and methanol.

Coupling: A solution of Nps-amino acid (4 eq), coupling reagent such as HBTU, HATU, HDTU, BOP, (6 eq), and HOBt or HOoBt, (6 eq), lutidene (8 eq), DIEA (8 eq) in NMP (1.5 ml), was allowed to stand for 5 min (for activation) and then added to the reaction vessel. The reaction mixture was vortexed for 1 h, filtered and then the resin was washed with NMP, DCM and methanol.

Nps cleavage: The resin was treated with 3% DCA in 1M thioacetamide for 25 min and then washed with NMP, methanol, and DCM.

The free amine was determined by Caiser test.

Side chains deprotection: The peptide on resin was treated with 1M tetrabutyl ammonium fluoride for 30 min, filtered and then washed with NMP, methanol, and DCM.

Cleavage from resin: (1) TGA Resin was treated with trifluoroacetic acid for 3 h, and the peptide was precipitated by ether; or (2) PAM Resin was treated with aqueous ammonium solution for 18 h at 55° C., the solution evaporated in vacuo and then lyophilized. The final peptide chain was determined by MS-ES.

### Preparation of Nucleotides

(1) Preparation of 5′O-DMT protected nucleoside (A^{Bz}, C^{Bz}, T)

Protected nucleoside was dried by co-evaporation with dry pyridine three times. To a stirred suspension of 5 mmol of nucleoside in pyridine, a solution of 1.7 g (5 mmol) dimethoxytrityl chloride in 10 ml pyridine was added dropwise over a period of 60 min. The reaction mixture was left for 4 h at room temperature, cooled to 0° C. (ice/water bath), quenched with 20 ml of 5% NaHCO₃, and extracted three times with ethyl acetate. The organic layer was dried (MgSO₄), concentrated in a vacuum, and the residue was co-evaporated with toluene. The gum oil obtained was dissolved in a minimum amount of dichloromethane and added dropwise to ethylene:petroleum ether (75:25) with stirring. After 20 min, pure 5′O-DMT-nucleoside was precipitated from the solution, filtered, and dried.

(II) Preparation of 3′-hydrogen Phosphonate

To 20 ml dry DCM were added 0.1 ml (1.13 mmol) phosphorous trichloride, 0.7 g (9 eq) of dry imidazole, and 0.45 ml of triethylamine at room temperature under N₂. After 1 h a mixture of 1 mmol of 5′O-DMT nucleoside and 0.08 g (1 mmol) tetrazole were added over a period of 10 min. The reaction mixture was stirred for an additional 2 h followed by addition of 20 ml water, and then extraction. The organic layer was dried (MgSO₄) and evaporated under reduced pressure. The resultant solid was collected, dried under vacuum, and characterized by ¹H and ³¹P NMR spectroscopy.

### 5′-Dimethoxytrityl-3′-H-phosphonate-2′-Deoxybenzoyl Adenine (55)

Yield 0.649 g (92%).

¹H NMR (DMSO-d₆, δ): 11.23 (br, 1H, NH of base); 8.62 (s, 1H, H8); 8.21-7.55 (m, 5H, aromatic of benzyl); 7.38-7.16 (m, 9H, aromatic of DMT); 6.71-6.69 (d, 4H, aromatic of DMT); 6.45 (t, 1H, H₁′); 5.76 (s, H₃′—P, J¹_{H-P}=585.2 Hz); 4.83 (m, 1H, H₃′); 4.21 (m, 1H, H₄′); 3.69 (s, 6H, O—CH₃ of DMT); 3.34 (m, 2H, H₅′ and H₅″); 3.12 (m, 1H, H₂′); 2.56 (m, 1H, H₂″).

³¹P NMR ¹H coupled (DMSO-d₆, δ): 0.982 (dd, H—P₃′, J¹_{P-H}=585.3 Hz; J³_{P-H}=8.5 Hz).

### 5′-Dimethoxytrityl-3′-H-phosphonate-2′-Deoxybenzoyl Cytosine (56)

Yield 0.627 g (90%).

¹H NMR (DMSO-d₆, δ): 11.31 (dr, 1H, NH of base); 8.21 (d, 1H, H6); 8.01-7.45 (m, 5H, aromatic of benzyl); 7.41-7.23 (m, 9H, aromatic of DMT); 7.12 (d, 1H, H₅); 6.75 (d, 4H, aromatic of DMT); 6.18 (t, 1H, H₁′); 5.67 (s, H₃′—P, J¹_{H-P}=585.4 Hz); 4.15 (m, 1H, H₄′), 3.72 (s, 6H, O—CH3 of DMT); 3.32 (m, 2H, H₅′ and H₅″); 2.26 (m, 1H, H₂″); 2.25 (m, 1H, H₂′) ³¹P NMR ¹H coupled (DMSO-d₆, □): 1.10 (dd, H—P₃′, J¹_{P-H}=586.5 Hz; J³_{P-H}=7.89 Hz).

### 5′-Dimethoxytrityl-3′-H-phosphonate-2′-Deoxy Thymine (57)

Yield 0.578 g (95%).

¹H NMR (CDCl₃-d₁, δ): 11.28 (br, 1H, NH of base); 7.48 (s, 1H, H6); 7.41-7.22 (m, 9H, aromatic of DMT); 6.8 (d, 4H, aromatic of DMT); 6.38 (t, 1H, H₁′); 5.65 (s, 1H, H₃′—P, J¹_{H-P}=585.2 Hz); 4.73 (m, 1H, H₃′); 4.15 (m, 1H, H₄′); 3.72 (s, 6H, O—CH3 of DMT); 3.2 (m, 2H, H₅′ and H₅″); 2.43-2.29 (m, 2H, H₂′ and E₂″); 1.37 (s, 3H, CH3 of base). ³¹P NMR ¹H coupled (CDCl₃-d₁, δⁱ): 1.01 (dd, H—P₃′, J¹_{P-H}=585.3 Hz; J³_{P-H}=8.5 Hz).

### Oligonucleotide Chain Elongation

Nucleotide building blocks were assembled on hydroxyl group of homoserine attached to PAM resin (see Scheme 18).

Coupling step: Each cycle of chain elongation consisted of detritylation, coupling (0.05 m monomer, 0.1-0.2 M of coupling reagent, DIEA (6 eq) and NMP (1 ml)) washing (NMP, DCM), capping and washing (NMP, methanol and DCM).

DMT cleavage: The resin was treated with 6% DCA in acetonitrile for 20 min, and then washed with NMP, acetonitrile and DCM.

The extent of the coupling was determined by the orange color formed by the free DMT.

Cleavage from resin and nucleobases deprotection: After oxidation, the resin was treated with aqueous ammonia solution for 18 h at 55° C. After the filtration, the solution was then evaporated to get the ODN chain, purified on HPLC and the molecular weight was verified by MS-ES.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

### REFERENCES

1. Phillips, M. I. & Gyurko, R. In-Vivo Applications of Antisense Oligonucleotides for Peptide Research. Regulatory Peptides 59, 131-141 (1995).
2. Pirollo, K. F., Rait, A., Sleer, L. S. & Chang, E. H. Antisense therapeutics: from theory to clinical practice. Pharmacology & Therapeutics 99, 55-77 (2003).
3. Morris, M. C., Vidal, P., Chaloin, L., Heitz, F. & Divita, G. A new peptide vector for efficient delivery of oligonucleotides into mammalian cells. Nucleic Acids Research 25, 2730-2736 (1997).
4. Ha, H. C. et al. The natural polyamine spermine functions directly as a free radical scavenger. Proceedings of the National Academy of Sciences of the United States of America 95, 11140-11145 (1998).
5. Pichon, C. et al. Cytosolic and nuclear delivery of oligonucleotides mediated by an amphiphilic anionic peptide. Antisense & Nucleic Acid Drug Development 7, 335-343 (1997).
6. Parente, R. A., Nadasdi, L., Subbarao, N. K. & Szoka, F. C. Association of a Ph-Sensitive Peptide with Membrane-Vesicles—Role of Amino-Acid-Sequence. Biochemistiy 29, 8713-8719 (1990).
7. Wyman, T. B. et al. Design, synthesis, and characterization of a cationic peptide that binds to nucleic acids and permeabilizes bilayers. Biochemistry 36, 3008-3017 (1997).
8. Deshpande, D., ToledoVelasquez, D., Thakkar, D., Liang, W. W. & Rojanasakul, Y. Enhanced cellular uptake of oligonucleotides by EGF receptor-mediated endocytosis in A549 cells. Pharmaceutical Research 13, 57-61 (1996).
9. Morris, M. C., Chaloin, L., Mery, J., Heitz, F. & Divita, G. A novel potent strategy for gene delivery using a single peptide vector as a carrier. Nucleic Acids Research 27, 3510-3517 (1999).
10. White, J. M. Viral and Cellular Membrane-Fusion Proteins. Annual Review of Physiology 52, 675-697 (1990).
11. Kielian, M. & Jungerwirth, S. Mechanisms of Enveloped Virus Entry into Cells. Molecular Biology & Medicine 7, 17-31 (1990).
12. Fujii, G., Selsted, M. E. & Eisenberg, D. Defensins Promote Fusion and Lysis of Negatively Charged Membranes. Protein Science 2, 13011312—(1993).
13. Murata, M., Takahashi, S., Kagiwada, S., Suzuki, A. & Ohnishi, S. Ph-Dependent Membrane-Fusion and Vesiculation of Phospholipid Large Unilamellar Vesicles Induced by Amphiphilic Anionic and Cationic Peptides. Biochemistry 31, 1986-1992 (1992).
14. Ho, A., Schwarze, S. R., Mermelstein, S. J., Waksman, G. & Dowdy, S. F. Synthetic protein transduction domains: Enhanced transduction potential in vitro and in vivo. Cancer Research 61, 474-477 (2001).
15. Chaloin, L. et al. Improvement of porphyrin cellular delivery and activity by conjugation to a carrier peptide. Bioconjugate Chemistry 12, 691-700 (2001).
16. Lin, Y. Z., Yao, S. Y., Veach, R. A., Torgerson, T. R. & Hawiger, J. Inhibition of Nuclear Translocation of Transcription Factor Nf-Kappa-B by a Synthetic Peptide-Containing a Cell Membrane-Permeable Motif and Nuclear-Localization Sequence. Journal of Biological Chemistry 270, 14255-14258 (1995).
17. Derossi, D., Joliot, A. H., Chassaing, G. & Prochiantz, A. The 3rd Helix of the Antennapedia Homeodomain Translocates through Biological-Membranes. Journal of Biological Chemistry 269, 10444-10450 (1994).
18. Derossi, D., Chassaing, G. & Prochiantz, A. Trojan peptides: the penetratin system for intracellular delivery. Trends in Cell Biology (1998) 87-84, 8.
19 Fawell, S. et al. Tat-Mediated Delivery of Heterologous Proteins into Cells. Proceedings of the National Academy of Sciences of the United States of America 91, 664-668 (1994).
20. Chen, L. L. et al. Increased Cellular Uptake of the Human Immunodeficiency Virus-1 Tat Protein after Modification with Biotin. Analytical Biochemistry 227, 168-175 (1995).
21. Dokka, S., Toledo-Velasquez, D., Shi, X. L., Wang, L. Y. & Rojanasakul, Y. Cellular delivery of oligonucleotides by synthetic import peptide carrier. Pharmaceutical Research 14, 1759-1764 (1997).
22. Bachmann, A. S., Surovoy, A., Jung, G. & Moelling, K. Integrin receptor-targeted transfer peptides for efficient delivery of antisense oligodeoxynucleotides. Journal of Molecular Medicine-Jmm 76, 126-132 (1998).23. Citro, G. et al. Inhibition of Leukemia-Cell Proliferation by Receptor-Mediated Uptake of C-Myb Antisense Oligodeoxynucleotides. Proceedings of the National Academy of Sciences of the United States of America 89, 7031-7035 (1992).
24. Reubi, J. C. et al. SST3-selective potent peptidic somatostatin receptor antagonists. Proceedings of the National Academy of Sciences of the United States of America 97, 13973-13978 (2000).
25. Wagner, E. Application of membrane-active peptides for nonviral gene delivery. Advanced Drug Delivery Reviews 38, 279-289 (1999).
26. Melino, S. et al. Zn2+ ions selectively induce antimicrobial salivary peptide histatin-5 to fuse negatively charged vesicles. Identification and characterization of a zinc-binding motif present in the functional domain. Biochemistry 38, 9626-9633 (1999).
27. Glaser, R. W., Grune, M., Wandelt, C. & Ulrich, A. S. Structure analysis of a fusogenic peptide sequence from the sea urchin fertilization protein bindin. Biochemistry 38, 2560-2569 (1999).
28. Vogt, T. C. B. & Bechinger, B. The interactions of histidine-containing amphipathic helical peptide antibiotics with lipid bilayers—The effects of charges and pH. Journal of Biological Chemistry 274, 29115-29121 (1999).
29. Pichon, C. et al. Intracellular routing and inhibitory activity of oligonucleopeptides containing a KDEL motif. Molecular Pharmacology 51, 431-438 (1997).
30. Hughes, J. A., Aronsohn, A. I., Avrutskaya, A. V. & Juliano, R. L. Evaluation of adjuvants that enhance the effectiveness of antisense oligodeoxynucleotides. Pharmaceutical Research 13, 404-410 (1996).
31. Vives, E., Brodin, P. & Lebleu, B. A truncated HIV-1 Tat protein basic domain rapidly translocates through the plasma membrane and accumulates in the cell nucleus. Journal of Biological Chemistry 272, 16010-16017 (1997).
32. Elliott, G. & Ohare, P. Intercellular trafficking and protein delivery by a herpesvirus structural protein. Cell 88, 223-233 (1997).
33. Langel, U., Pooga, M., Kairane, C., Zilmer, M. & Bartfai, T. A galanin-mastoparan chimeric peptide activates the Na+,K+-ATPase and reverses its inhibition by ouabain. Regulatory Peptides 62, 47-52 (1996).
34. Derossi, D. et al. Cell internalization of the third helix of the antennapedia homeodomain is receptor-independent. Journal of Biological Chemistry 271, 18188-18193 (1996).
35. Mazel, M. et al. Doxorubicin-peptide conjugates overcome multidrug resistance. Anti-Cancer Drugs 12, 107-116 (2001).
36. Rousselle, C. et al. New advances in the transport of doxorubicin through the blood-brain barrier by a peptide vector-mediated strategy. Molecular Pharmacology 57, 679-686 (2000).
37. Bayley, H. Protein therapy—delivery guaranteed. Nature Biotechnology 17, 1066 (1999).
38. Bhorade, R., Weissleder, R., Nalcakoshi, T., Moore, A. & Tung, C. H. Macrocyclic chelators with paramagnetic cations are internalized into mammalian cells via a HIV-tat derived membrane translocation peptide. Bioconjugate Chemistry 11, 301-305 (2000).
39. Allinquant, B. et al Down-Regulation of Amyloid Precursor Protein Inhibits Neurite Outgrowth in-Vitro. Journal of Cell Biology 128, 919-927 (1995).
40. Astriab-Fisher, A., Sergueev, D. S., Fisher, M., Shaw, B. R. & Juliano, R. L. Antisense inhibition of P-glycoprotein expression using peptide-oligonucleotide conjugates. Biochemical Pharmacology 60, 83-90 (2000).
41. Troy, C. M., Derossi, D., Prochiantz, A., Greene, L. A. & Shelanski, M. L. Downregulation of Cu/Zn superoxide dismutase leads to cell death via the nitric oxide-peroxynitrite pathway. Journal of Neuroscience 16, 253-261 (1996).
42. Hughes, J. et al. Methods in Enzymology 313, 342 (1999).
43. de la Torre, B. G., Albericio, F., Saison-Behmoaras, E., Bachi, A. & Eritja, R. Synthesis and binding properties of oligonucleotides carrying nuclear localization sequences. Bioconjugate Chemistry 10, 1005-1012 (1999).
44. Eritja, R., Pons, A., Escarceller, M., Giralt, E. & Albericio, F. Synthesis of Defined Peptide-Oligonucleotide Hybrids Containing a Nuclear Transport Signal Sequence. Tetrahedron 47, 4113-4120 (1991).
45. Reed, M. W., Fraga, D., Schwartz, D. E., Scholler, J. & Hinrichsen, R. D. Synthesis and Evaluation of Nuclear Targeting Peptide Antisense Oligodeoxynucleotide Conjugates. Bioconjugate Chemistry 6, 101-108 (1995).
46. Branden, L. J., Mohamed, A. J. & Smith, C. I. E. A peptide nucleic acid-nuclear localization signal fusion that mediates nuclear transport of DNA. Nature Biotechnology 17, 784-787 (1999).
47. de la Torre, B. G. et al. Stepwise Solid-Phase Synthesis of Oligonucleotide-Peptide Hybrids. Tetrahedron Letters 35, 2733-2736 (1994).
48. Bruick, R. K., Dawson, P. E., Kent, S. B., Usman, N. & Joyce, G. F. Template-directed ligation of peptides to oligonucleotides. Chemistry & Biology 3, 49-56 (1996).
49. Mitchell, D. J., Kim, D. T., Steinman, L., Fathman, C. G. & Rothbard, J. B. Polyarginine enters cells more efficiently than other polycationic homopolymers. Journal of Peptide Research 56, 318-325 (2000).
50. Futaki, S. et al. Arginine-rich peptides—An abundant source of membrane-permeable peptides having potential as carriers for intracellular protein delivery. Journal of Biological Chemistry 276, 5836-5840 (2001).
51. Wagner, E., Plank, C., Zatloukal, K., Cotten, M. & Birnstiel, M. L. Influenza-Virus Hemagglutinin-Ha-2 N-Terminal Fusogenic Peptides Augment Gene-Transfer by Transferrin Polylysine DNA Complexes—toward a Synthetic Virus-Like Gene-Transfer Vehicle. Proceedings of the National Academy of Sciences of the United States of America 89, 7934-7938 (1992)
52. Midoux, P. et al. Specific Gene-Transfer Mediated by Lactosylated Poly-L-Lysine into Hepatoma-Cells. Nucleic Acids Research 21, 871-878 (1993).
53. Plank, C., Oberhauser, B., Mechtler, K., Koch, C. & Wagner, E. The Influence of Endosome-Disruptive Peptides on Gene-Transfer Using Synthetic Virus-Like Gene-Transfer Systems. Journal of Biological Chemistry 269, 12918-12924 (1994).
54. Demspsey, C. E. Biochemica Biophisica Acta 1031, 143 (1990).
55. Fischer, P. M. et al. Structure-activity relationship of truncated and substituted analogues of the intracellular delivery vector Penetratin. Journal of Peptide Research 55, 163-172 (2000).
56. Pichon, C., Goncalves, C. & Midoux, P. Histidine-rich peptides and polymers for nucleic acids delivery A dvanced Drug Delivery Reviews 53, 75-94 (2001).
57 Harbottle, R. P. et al. An RGD-oligolysine peptide: A prototype construct for integrin-mediated gene delivery. Human Gene Therapy 9, 1037-1047 (1998).
58 Colin, M. et al. Liposomes enhance delivery and expression of an RGD-oligolysine gene transfer vector in human tracheal cells. Gene Therapy 5, 1488-1498 (1998).
59. Vaysse, L., Burgelin, I., Merlio, J. P. & Arveiler, B. Improved transfection using epithelial cell line-selected ligands and fusogenic peptides. Biochimica Et Biophysica Acta-General Subjects 1475, 369-376 (2000).
60. Shewring, L. et al. A nonviral vector system for efficient gene transfer to corneal endothelial cells via membrane integrin. Transplantation 64, 763-769 (1997).
61. Collins, L., Sawyer, G. J., Zhang, X. H., Gustafsson, K. & Fabre, J. W. In vitro investigation of factors important for the delivery of an integrin-targeted nonviral DNA vector in organ transplantation. Transplantation 69, 1168-1176 (2000).
62. Li, J. M., Collins, L., Zhang, X. H., Gustafsson, K. & Fabre, J. W. Efficient gene delivery to vascular smooth muscle cells using a nontoxic, synthetic peptide vector system targeted to membrane integrins: A first step toward the gene therapy of chronic rejection. Transplantation 70, 1616-1624 (2000).
63. Soukchareun, S., Tregear, G. W. & Haralambidis, J. Preparation and Characterization of Antisense Oligonucleotide Peptide Hybrids Containing Viral Fusion Peptides. Bioconjugate Chemistry 6, 43-53 (1995).
64. Soukchareun, S., Haralambidis, J. & Tregear, G. Use of N-alpha-Fmoc-cysteine(S-thiobutyl) derivatized oligodeoxynucleotides for the preparation of oligodeoxynucleotide-peptide hybrid molecules. Bioconjugate Chemistry 9, 466-475 (1998).
65. Bongartz, J. P., Aubertin, A. M., Milhaud, P. G. & Lebleu, B. Improved Biological-Activity of Antisense Oligonucleotides Conjugated to a Fusogenic Peptide. Nucleic Acids Research 22, 4681-4688 (1994).
66. Arar, K., Aubertin, A. M., Roche, A. C., Monsigny, M. & Mayer, R. Synthesis and Antiviral Activity of Peptide-Oligonucleotide Conjugates Prepared by Using N-Alpha-(Bromoacetyl)Peptides. Bioconjugate Chemistry 6, 573-577 (1995).
67. Arar, K., Monsigny, M. & Mayer, R. Synthesis of Oligonucleotide-Peptide Conjugates Containing a Kdel Signal Sequence. Tetrahedron Letters 34, 8087-8090 (1993).
68. Robles, J. et al. Synthesis and enzymatic stability of phosphodiester-linked peptide—Oligonucleotide hybrids. Bioconjugate Chemistry 8, 785-788 (1997).
69 Chen, C. P. et al. Synthesis of antisense oligonucleotide-peptide conjugate targeting to GLUT-1 in HepG-2 and MCF-7 cells. Bioconjugate Chemistry 13, 525-529 (2002).
70. De Napoli, L. et al. A new solid-phase synthesis of oligonucleotides 3′-conjugated with peptides. Bioorganic & Medicinal Chemistry 7, 395-400 (1999).
71. Chen, C. P. et al. A concise method for the preparation of peptide and arginine-rich peptide-conjugated antisense oligonucleotide. Bioconjugate Chemistry 14, 532-538 (2003).
72. Schwope, I., Bleczinski, C. F. & Richert, C. Synthesis of 3′,5′-dipeptidyl oligonucleotides. Journal of Organic Chemistry 64, 4749-4761 (1999).
73. Antopolsky, M. & Azhayev, A. Stepwise solid-phase synthesis of peptide-oligonucleotide phosphorothioate conjugates employing Fmoc peptide chemistry. Tetrahedron Letters 41, 9113-9117 (2000).
74. Stetsenko, D. A., Malakhov, A. D. & Gait, M. J. Total stepwise solid-phase synthesis of oligonucleotide-(3′->N)-peptide conjugates. Organic Letters 4, 3259-3262 (2002).
75. Basu, S. & Wickstrom, E. Solid-Phase Synthesis of a D-Peptide-Phosphorothioate Oligodeoxynucleotide Conjugate from 2 Arms of a Polyethylene Glycol-Polystyrene Support. Tetrahedron Letters 36, 4943-4946 (1995).
76. Bergmann, F. & Bannwarth, W. Solid-Phase Synthesis of Directly Linked Peptide-Oligodeoxynucleotide Hybrids Using Standard Synthesis Protocols. Tetrahedron Letters 36, 1839-1842 (1995).
77. Juby, C. D., Richardson, C. D. & Brousseau, R. Facile Preparation of 3′-Oligonucleotide-Peptide Conjugates. Tetrahedron Letters 32, 879-882 (1991)
78. Antopolsky, M. & Azhayev, A. Stepwise solid-phase synthesis of peptide-oligonucleotide conjugates on new solid supports. Helvetica Chimica Acta 82, 2130-2140 (1999).
79. Antopolsky, M., Azhayeva, E., Tengvall, U. & Azhayev, A. Towards a general method for the stepwise solid-phase synthesis of peptide-oligonucleotide conjugates. Tetrahedron Letters 43, 527-530 (2002).
80. Zubin, E. M. et al. Oligonucleotide-peptide conjugates as potential antisense agents. Febs Letters 456, 59-62 (1999).
81. Mier, W., Eritja, R., Mohammed, A., Haberkom, U. & Eisenhut, M. Preparation and evaluation of tumor-targeting peptide-oligonucleotide conjugates. Bioconjugate Chemistry 11, 855-860 (2000).
82. Kubo, T., Morikawa, M., Ohba, H. & Fujii, M. Synthesis of DNA-peptide conjugates by solid-phase fragment condensation. Organic Letters 5, 2623-2626 (2003).
83. Stetsenko, D. A. & Gait, M. J. Efficient conjugation of peptides to oligonucleotides by “native ligation”. Journal of Organic Chemistry 65, 4900-4908 (2000).
84. Tengvall, U., Auriola, S., Antopolsky, M., Azhayev, A. & Biegelman, L. Characterization of antisense oligonucleotide-peptide conjugates with negative ionization electrospray mass spectrometry and liquid chromatography-mass spectrometry. Journal of Pharmaceutical and Biomedical Analysis 32, 581-590 (2003).
85. Antopolsky, M. et al. Peptide-oligonucleotide phosphorothioate conjugates with membrane translocation and nuclear localization properties. Bioconjugate Chemistry 10, 598-606 (1999).
86. Ollivier, N. et al. Synthesis of oligonucleotide-peptide conjugates using hydrazone chemical ligation. Tetrahedron Letters 43, 997-999 (2002).
87. Zatsepin, T. S. et al. Synthesis of peptide-oligonucleotide conjugates with single and multiple peptides attached to 2′-aldehydes through thiazolidine, oxime, and hydrazine linkages. Bioconjugate Chemistry 13, 822-830 (2002).
88. Mitchell, A. R., Erickson, B. W., Ryabtsev, M. N., Hodges, R. S. & Merrifield, R. B. Tert-Butoxycarbonylaminoacyl-4-(Oxymethyl)-Phenylacetamidomethyl-Resin, a More Acid-Resistant Support for Solid-Phase Peptide-Synthesis. Journal of the American Chemical Society 98, 7357-7362 (1976).
89. Mitchell, A. R., Kent, S. B. H., Engelhard, M. & Merrifield, R. B. New Synthetic Route to Tert-Butyloxycarbonylaminoacyl-4-(Oxymethyl)Phenylacetamidomethyl-Resin, an Improved Support for Solid-Phase Peptide-Synthesis. Journal of Organic Chemistry 43, 2845-2852 (1978).
90. Matsueda, G. R. & Stewart, J. M. A P-Methylbenzhydrylamine Resin for Improved Solid-Phase Synthesis of Peptide Amides. Peptides 2, 45-50 (1981).
91. Sakakiba. S, Shimonis. Y, Kishida, Y., Okada, M. & Sugihara, H. Use of Anhydrous Hydrogen Fluoride in Peptide Synthesis. I. Behavior of Various Protective Groups in Anhydrous Hydrogen Fluoride. Bulletin of the Chemical Society of Japan 40, 2164-& (1967).
92. Yajima, H., Fujii, N., Ogawa, H. & Kawatani, H. Trifluoromethanesulphonic Acid, as a Deprotecting Reagent in Peptide Chemistry. Journal of the Chemical Society-Chemical Communications, 8, 107-108 (1974)
93. Fujii, N. et al. Trimethylsilyl Trifluoromethanesulfonate as a Useful Deprotecting Reagent in Both Solution and Solid-Phase Peptide Syntheses. Journal of the Chemical Society-Chemical Communications, 274-275 (1987).
94. Degrado, W. F. & Kaiser, E. T. Polymer-Bound Oxime Esters as Supports for Solid-Phase Peptide-Synthesis—Preparation of Protected Peptide-Fragments. Journal of Organic Chemistry 45, 1295-1300 (1980).
95. Barany, G. & Albericio, F. A 3-Dimensional Orthogonal Protection Scheme for Solid-Phase Peptide-Synthesis under Mild Conditions. Journal of the American Chemical Society 107, 4936-4942 (1985).
96. Kunz, H. & Dombo, B. Solid-Phase Synthesis of Peptides and Glycopeptides on Polymeric Supports with Allylic Anchor Groups. Angewandte Chemie-International Edition in English 27, 711-713 (1988).
97. Tarbell, D. S., Yamamoto, Y. & Pope, B. M. New Method to Prepare N Tert Butoxycarbonyl Derivatives and Corresponding Sulfur Analogs from Di Tert Butyl Dicarbonate or Di Tert Butyl Dithiol Dicarbonates and Amino-Acids. Proceedings of the National Academy of Sciences of the United States of America 69, 730-& (1972).
98. Chang, C. D. & Meienhofer, J. Solid-Phase Peptide-Synthesis Using Mild Base Cleavage of Nalpha-Fluorenylmethyloxycarbonylamino Acids, Exemplified by a Synthesis of Dihydrosomatostatin. International Journal of Peptide and Protein Research 11, 246-249 (1978).
99. Atherton, E. et al. Mild Procedure for Solid-Phase Peptide-Synthesis—Use of “Fluorenylmethoxycarbonylamino-Acids. Journal of the Chemical Society-Chemical Communications, 537-539 (1978).
100. Wang, S. S. Para-Alkoxybenzyl Alcohol Resin and Para-Alkoxybenzyloxycarbonylhydrazide Resin for Solid-Phase Synthesis of Protected Peptide Fragments. Journal of the American Chemical Society 95, 1328-1333 (1973).
101. Barlos, K. et al. Synthesis of Protected Peptide-Fragments Using Substituted Triphenylmethyl Resins. Tetrahedron Letters 30, 3943-3946 (1989).
102. Rink, H. Solid-Phase Synthesis of Protected Peptide-Fragments Using a Trialkoxy-Diphenyl-Methylester Resin. Tetrahedron Letters 28, 3787-3790 (1987).
103. Horiki, K. Amino-Acids and Peptides .3. Behavior of Acylated 1-Hydroxybenzotriazole. Tetrahedron Letters, 1897-1900 (1977).
104. Sheehan, J. C. & Hess, G. P. A New Method of Forming Peptide Bonds. Journal of the American Chemical Society 77, 1067-1068 (1955).
105. Sheehan, J. C., Cruickshank, P. A. & Boshart, G. L. A Convenient Synthesis of Water-Soluble Carbodiimides. Journal of Organic Chemistry 26, 2525-2528 (1961).
106. Carpino, L. A. 1-Hydroxy-7-Azabenzotriazole—an Efficient Peptide Coupling Additive. Journal of the American Chemical Society 115, 4397-4398 (1993).
107. Fields, C. G., Lloyd, D. H., Macdonald, R. L., Otteson, K. M. & Noble, R. L. HBTU Activation for Automated Fmoc Solid-Phase Peptide Synthesis. Peptide Research 4, 95-101 (1991).
108. Castro, B., Dormoy, J. R., Evin, G. & Selve, C. Reactions of Peptide Bond .4. Benzotriazonyl-N-Oxytridimelthylamino Phosphonium Hexafluorophosphate (Bop). Tetrahedron Letters, 1219-1222 (1975).
109. Coste, J., Lenguyen, D. & Castro, B. Pybop—a New Peptide Coupling Reagent Devoid of Toxic by-Product. Tetrahedron Letters 31, 205-208 (1990).
110. Haralambidis, J. et al. The Preparation of Polyamide-Oligonucleotide Probes Containing Multiple Nonradioactive Labels. Nucleic Acids Research 18, 501-505 (1990).
111. Schaller, H., Weimann, G., Khorana, H. G. & Lerch, B. Studies on Polynucleotides .24. Stepwise Synthesis of Specific Deoxyribopolynucleotides (4)—Protected Derivatives of Deoxyribonucleosides and New Syntheses of Deoxyribonucleoside-3 Phosphates. Journal of the American Chemical Society 85, 3821-& (1963).
112. Khorana, H. G. Pure Applide Chemistry 17, 349 (1968).
113. Agarwal, K. L., Cashion, P. J., Yamazaki, A. & Khorana, H. G. Chemical Synthesis of Polynucleotides. Angewandte Chemie-International Edition 11, 451-& (1972).
114. Smith, M., Rammler, D. H., Goldberg, I. H. & Khorana, H. G. Studies on Polynucleotides. XIV. Specific Synthesis of the C₃′-C₅′ Interribonucleotide Linkage. Syntheses of Uridylyl-(3′-5′)-Uridine and Uridylyl-(3′-5′)-Adenosine. Journal of the American Chemical Society 84, 430-440 (1962).
115. Adams, S. P., Kavka, K. S., Wykes, E. J., Holder, S. B. & Galluppi, G. R. Hindered Dialkylamino Nucleoside Phosphite Reagents in the Synthesis of 2 DNA 51-Mers. Journal of the American Chemical Society 105, 661-663 (1983).
116. Khorana, H. G., Tener, G. M., Motfatt, J. G. & Pol, E. H. Chemistry and Industry, 1523 (1956).
117. Khorana, H. G., Razzell, W. E., Gilham, P. T., Tener, G. M. & Pol, E. H. Synthesis of Dideoxyribonucleotides. Journal of the American Chemical Society 79, 1002-1003 (1957).
118. Jacob, T. M. & Khorana, H. G. Studies on Polynucleotides .30. Comparative Study of Reagents for Synthesis of C3-C5 Internucleotidic Linkage. Journal of the American Chemical Society 86, 1630-& (1964).
119. Lohrmann. R & Khorana, H. G. Studies on Polynucleotides .52. Use of 2 4 6-Triisopropylbenzenesulfonyl Chloride for Synthesis of Internucleotide Bonds. Journal of the American Chemical Society 88, 829-& (1966).
120. Gilham, P. T. & Tener, G. M. Chemistry and Industry, 245 (1956).
121. McBride, L. J. & Caruthers, M. H. Nucleotide Chemistry .10. An Investigation of Several Deoxynucleoside Phosphoramidites Useful for Synthesizing Deoxyoligonucleotides. Tetrahedron Letters 24, 245-248 (1983).
122. Beaucage, S. L. & Caruthers, M. H. Deoxynucleoside Phosphoramidites—a New Class of Key Intermediates for Deoxypolynucleotide Synthesis. Tetrahedron Letters22, 1859-1862 (1981).
123. Garegg, P. J. et al. Nucleoside H-Phosphonates .3. Chemical Synthesis of Oligodeoxyribonucleotides by the Hydrogenphosphonate Approach. Tetrahedron Letters 27, 4051-4054 (1986).
124. Garegg, P. J. et al. Nucleoside H-Phosphonates .4. Automated Solid-Phase Synthesis of Oligoribonucleotides by the Hydrogenphosphonate Approach. Tetrahedron Letters 27, 4055-4058 (1986).
125. Zervas, L., Borovas, D. & Gazis, E. New Methods in Peptide Synthesis .1. Tritylsulfenyl and O-Nitrophenylsulfenyl Groups as N-Protecting Groups. Journal of the American Chemical Society 85, 3660-& (1963).
126. Belshaw, R. J., Adamson, J. G. & Lajoie, G. A. Synthetic Communication 22, 1001 (1992).
127. de Groot, F. M., de Bart, A. C., Verheijen, J. H. & Scheeren, H. W. Synthesis and Biological Evaluation of Novel Prodrugs of Anthracyclines for Selective Activation by the Tumor-Associated Protease Plasmin. Journal of Medicinal Chemistry 42, 5277-5283 (1999).
128. Alvarez, K., Tworkowski, I., Vasseur, J-J., hnbach, J.-L. & Rayner, B. A reinvestigation of sulfenyl groups as amino protecting groups for the synthesis of oligonucleotides. Nucleosides & Nucleotides, 1998, 17, 365-378.
129. Ueki Massald & Amemiya Masahide. Removal of Fmoc group with tetrabutylammonium fluoride. Tetrahedron Lett., 1987, 28, 6617-6620; Jiang J., Li, W. & Joullie M. M. Selective Removal Fmoc groups under mild conditions. Synth. Commun., 1994, 24, 187-196.

1. A method for the preparation of a peptide-oligonucleotide conjugate (POC), said method comprising the steps of:
a. providing a first N-α-o-nitrophenyl sulphenyl (N-α-Nps)-protected amino acid or a first nucleotide;
b. coupling, in any order, at least a second N-α-Nps-protected amino acid and/or at least a second nucleotide to said first N-α-Nps-protected amino acid or said first nucleotide; and
c. repeating step (b) as necessary, so as to form a peptide-oligonucleotide conjugate having at least one amino acid-nucleotide bond;
wherein each coupling step is conducted in the presence of a coupling reagent compatible with peptide synthesis; and
wherein said N-α-Nps protecting group is removed prior to each amino acid-amino acid coupling step using thioacetamide in the presence of dichloroacetic acid.

2. The method according to claim 1, wherein said coupling reagent is selected from the group consisting of 1-hydroxybenzotriazole (HOBt), 3-hydroxy-3,4-dihydro-1,2,3-benzotriazine-4-one (HOOBt), N-hydroxysuccinimide (NHS), dicyclohexylcarbodiimide (DCC), diisopropylcarbodiimide (DIC), 1-ethyl-3-(3-dimethylaminopropyl)-carbodiimide (EDAC), 2-(1H-7-azabenztriazol-1-yl)-1,1,3,3-tetramethyluronium hexafluoro phosphate (HATU), 2-(1H-benzotriazol-1-yl)-1,1,3,3-tetramethyluronium hexafluorophosphate (HBTU), 3,4-dihydro-1,2,3-benzotriazin-4-one-3-oxy-tetramethyluronium hexafluorophosphate (HDTU), benzotriazol-1-yloxytris(dimethylamino)phosphonium hexafluoro phosphate (BOP), benzotriazol-1-yloxytris-(pyrrolidino)-pjosphonium hexafluoro phosphate (PyBop), (3,4-dihydro-1,2,3-benzotriazin-4-one-3-oxy) diethyl phosphate (DEPBt), 3,4-dihydro-1,2,3-benzotriazin-4-one-3-oxy-yloxytris-(pyrrolidino)-pjosphonium hexafluoro phosphate (PDOP), 2-(benzotriazol-1-yloxy)-1,3-dimethyl-2-pyrrolidin-1-yl-1,3,2-diazaphospholidinium hexafluorophosphonate (BOMP), 5-(1H-7-azabenzotriazol-1-yloxy)-3,4-dihydro-1-methyl 2H-pyrrolium hexachloroantimonate (AOMP), (1H-7-azabenzotriazol-1-yloxy)tris(dimethylamino) phosphonium hexafluoroposphate (AOP), 5-(1H-Benzotriazol-1-yl)-3,4-dihydro-1-methyl 2H-pyrrolium hexachloroantimonate: N-oxide (BDMP), 2-bromo-3-ethyl-4-methyl thiazolium tetrafluoroborate (BEMT), 2-bromo-1-ethyl pyridinium tetrafluoroborate (BEP), 2-bromo-1-ethyl pyridinium hexachloroantimonate (BEPH), N-(1H-benzotriazol-1-ylmethylene)-N-methylmethanaminium hexachloroantimonate N-oxide (BOMI), N,N′-bis(2-oxo-3-oxazolidinyl) phosphinic chloride (BOP-Cl), 1-(1H-benzotriazol-1-yloxy)phenylmethylene pyrrolidinium hexachloroantimonate (BPMP), 1,1,3,3-bis(tetramethylene) fluorouronium hexafluorophosphate (BTFFH), chloro(4-morphoino)methylene morpholinium hexafluorophosphate (CMMM), 2-chloro-1,3-dimethyl-1H-benzimidazolium hexafluorophosphate (CMBI), 2-fluoro-1-ethyl pyridinium tetrafluoroborate (FEP), 2-fluoro-1-ethyl pyridinium hexachloroantimonate (FEPH), 1-(1-pyrrolidinyl-1H-1,2,3-triazolo[4,5-b]pyridin-1-ylmethylene)pyrrolidinium hexafluorophosphate N-oxide (HAPyU), O-(1H-benzotriazol-1-yl)-N,N,N′,N;-bis(pentamethylene)uronium hexafluorophosphate (HBPipU), O-(1H-benzotriazol-1-yl)-N,N,N0,N0-bis(tetramethylene)urinium hexafluorophosphate (HBPyU), (1H-7-azabenzotriazol-1-yloxy)tris(pyrrolidino)phosphonium hexafluorophosphate (PyAOP), bromotripyrrolidinophosphonium hexafluorophosphate (PyBrop), chlorotripyrrolidinophosphonium hexafluorophosphate (PyCloP), 1,1,3,3-bis(tetramethylene) chlorouronium hexafluorophosphate (PyCIU), tetramethylfluoromamidinium hexafluorophosphate (TFFH), triphosgene, triazine-based reagents, bis(2-chlorophenyl) phosphorochloridate, diphenyl phosphorochloridate, diphenyl phosphoroazide (DPPA), and any combination thereof.

3.-14. (canceled)

15. The method according to claim 1, wherein said N-α-Nps-protected amino acid is a side-chain protected amino acid.

16. (canceled)

17. The method according to claim 15, wherein said side chain protecting group is a silyl protecting group of the formula (R)₄Si wherein each R is independently of the other an unsubstituted or substituted alkyl, alkylaryl, aryl, oxyalkyl, oxyalkylaryl, or oxyaryl.

18. The method according to claim 15, wherein said side chain protecting group is represented by the structure: 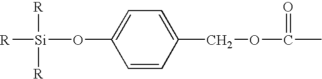
wherein each R is independently of the other selected from the group consisting of an unsubstituted or substituted alkyl, alkylaryl, aryl, oxyalkyl, oxyalkylaryl and oxyaryl.

19. The method according to claim 18, wherein R is isopropyl.

20. The method according to claim 18, wherein said side-chain protected amino acid is prepared by coupling said side chain with a compound of the formula:

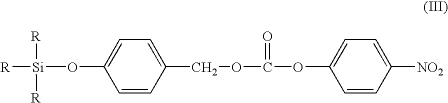

21. The method according to claim 15, wherein said side-chain protecting group is Fmoc.

22. The method according to claim 15, wherein said side-chain protecting group is an Fm ester.

23. The method according to claim 1, wherein each nucleotide-nucleotide coupling step is conducted by phosphate coupling, H-phosphonate coupling or phosphate coupling, or any combination thereof.

24. The method according to claim 1, wherein each nucleotide-nucleotide coupling step is conducted by H-phosphonate coupling.

25. The method according to claim 1, wherein said POC is prepared on a solid support.

26. The method according to claim 1, wherein said oligonucleotide is synthesized first.

27. The method according to claim 1, wherein said peptide is synthesized first.

28. The method according to claim 1, wherein said peptide and said oligonucleotide are synthesized in alternating sequences.

29. A method for the preparation of a peptide-oligonucleotide conjugate (POC), said method comprising the steps of:
a. providing a first N-α-o-nitrophenyl sulphenyl (N-α-Nps)-protected amino acid or a first nucleotide;
b. coupling, in any order, at least a second N-α-Nps-protected amino acid and/or at least a second nucleotide to said first N-α-Nps-protected amino acid or said first nucleotide; and
c. repeating step (b) as necessary, so as to form a peptide-oligonucleotide conjugate having at least one amino acid-nucleotide bond;
wherein each coupling step is conducted in the presence of a coupling reagent compatible with peptide synthesis;
wherein said N-α-Nps protecting group is removed prior to each amino acid-amino acid coupling step using thioacetamide in the presence of dichloroacetic acid; and
wherein each nucleotide-nucleotide coupling step is conducted by H-phosphonate coupling.

30.-54. (canceled)

55. A compound represented by the structure: 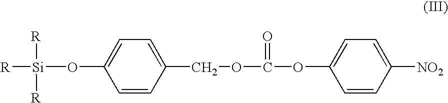
wherein each R is independently of the other selected from the group consisting of an unsubstituted or substituted alkyl, alkylaryl, aryl, oxyalkyl, oxyalkylaryl and oxyaryl.

56. The compound according to claim 55, wherein R is isopropyl.

57.-58. (canceled)

59. A side-chain protected amino acid represented by the structure: 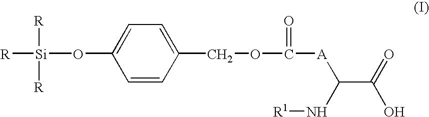
wherein
A represents a side chain residue of said amino acid;
R is independently selected from the group consisting of an unsubstituted or substituted alkyl, alkylaryl, aryl, oxyalkyl, oxyalkylaryl and oxyaryl; and
R¹ represents hydrogen or an amino protecting group.

60. The side-chain protected amino acid according to claim 59, wherein said amino acid is selected from the group consisting of arginine, lysine, aspartic acid, asparagine, glutamic acid, glutamine, histidine, cysteine, homocysteine, ornithine, serine, homoserine, threonine, homoarginine, citrulline and tyrosine.

61. The side-chain protected amino acid according to claim 59, wherein R¹ is o-nitrophenyl sulphenyl (Nps).

62. A method for preparing the side-chain protected amino acid of claim 59 comprising the step of reacting said amino acid with a compound of the formula: 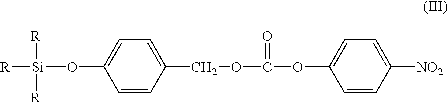
thereby forming said side-chain protected amino acid.

63. The method according to claim 62, wherein said amino acid is selected from the group consisting of arginine, lysine, aspartic acid, asparagine, glutamic acid, glutamine, histidine, cysteine, homocysteine, ornithine, serine, homoserine, threonine, homoarginine, citrulline and tyrosine.

64. The method according to claim 62, wherein R¹ is o-nitrophenyl sulphenyl (Nps).

65. A method for the preparation of a peptide-oligonucleotide conjugate (POC), said method comprising the step of:
performing at least one coupling between an α-amino protected amino acid and a nucleotide so as to form a peptide-oligonucleotide conjugate having at least one amino acid-nucleotide bond;
wherein said amino acid or nucleotide further comprise one or more orthogonal protecting groups where required;
wherein each coupling step is conducted in the presence of a coupling reagent compatible with peptide synthesis; and
wherein said α-amino protecting group is removed prior to each amino acid-amino acid coupling step using a deprotecting agent compatible with any one or more protecting groups present in the oligonucleotide-peptide conjugate.

66. The method according to claim 65, wherein said α-amino protecting group is N-α-o-nitrophenyl sulphenyl (N-αNps).

67. The method according to claim 65, wherein said α-amino protecting group is p-azidobenzyloxycarbonyl (ACBZ).

68. A method for the preparation of a peptide-oligonucleotide conjugate (POC), said method comprising the step of performing at least one coupling between an N-α-o-nitrophenyl sulphenyl (N-α-Nps) amino acid and a nucleotide so as to form a peptide-oligonucleotide conjugate having at least one amino acid-nucleotide bond;
wherein said N-α-Nps protected amino acid or nucleotide further comprise one or more orthogonal protecting groups where required;
wherein each coupling step is conducted in the presence of a coupling reagent compatible with peptide synthesis; and
wherein said N-α-Nps protected amino protecting group is removed prior to each amino acid-amino acid coupling step using a deprotecting agent compatible with any one or more protecting groups present in the oligonucleotide-peptide conjugate.
